(19) United States
(12) Patent Application Publication
(10) Pub. No.: US 2024/0355422 A1
(43) Pub. Date: Oct. 24, 2024

(54) ARTIFICIAL INTELLIGENCE (AI) BASED SELECTIVE SEQUENCING PLATFORM USING OXFORD NANOPORE TECHNOLOGY (ONT)

(71) Applicant: The Chinese University of Hong Kong, Hong Kong (CN)

(72) Inventors: Hao SUN, Hong Kong (CN); Xing ZHAO, Hong Kong (CN)

(21) Appl. No.: 18/640,124

(22) Filed: Apr. 19, 2024

Related U.S. Application Data
(60) Provisional application No. 63/496,985, filed on Apr. 19, 2023.

(51) Int. Cl. G16B 40/10; G01N 33/487; G06N 3/0442; H04L 67/12

(52) U.S. Cl. CPC G16B 40/10; G01N 33/48721; G06N 3/0442; H04L 67/12

(57) ABSTRACT

The subject invention pertains to DNA/RNA sequencing by measuring the characteristic electrical signal when DNA/RNA moves across a pore that is only nanometers in diameter. The sequencer allows reads to be rejected during sequencing in real time through selective sequencing by computational methods, reducing human labor and cost requirements. Electrical signals are used directly to decide if the sequenced reads are from selected genomic regions or from different sources. Provided Artificial Intelligence (AI) models are established by training deep learning neural networks using collected nanopore sequencing signals. The method can be easily integrated into existing nanopore sequencing infrastructure, offering real-time parallel molecule classification with the flexibility to meet the requirements of a variety of selective sequencing applications such as detecting pathogens in clinical samples, targeted gene panel for cancer diagnosis, as well as drug resistant bacterial screening, and numerous other applications.

### CROSS-REFERENCE TO RELATED APPLICATION

This application claims the benefit of U.S. Provisional Application Ser. No. 63/496,985, filed Apr. 19, 2023, which is hereby incorporated by reference in its entirety including any tables, figures, or drawings.

### BACKGROUND OF THE INVENTION

Nanopore sequencing is a novel sequencing technology introduced by Oxford Nanopore Technology (ONT). It is also known as Long Read Sequencing or Third Generation Sequencing (3GS) because of its ability to sequence DNA reads up to 100 kb (Jain, Koren et al. 2018). Nanopore sequencers measure the electrical signal fluctuations caused by a single DNA molecule moving across the pore which is only nanometers in diameter. Apart from obtaining the sequence of DNA nucleotides from the electrical signal. The same signal can be employed to detect DNA modifications such as DNA methylations as they exhibit different patterns than unmethylated DNA (Rand, Jain et al. 2017). Apart from DNA sequencing, there is also a protocol designed for direct RNA sequencing (DRS) which allows full-length native RNA molecules to be sequenced without introducing bias from PCR and reverse transcription. Hence, useful information such as RNA modifications and polyA tail length can only be investigated using DRS (Workman, Tang et al. 2019). DRS has been employed to investigate novel transcripts in various species such as human, yeast, Caenorhabditis elegans and plants (Zhao, Zhang et al. 2019, Roach, Sadowski et al. 2020, Vacca, Fiannaca et al. 2022, Mock, Braun et al. 2023). Compared to similar protocols such as Illumina-based sequencing methods, DRS has significantly longer maximum read lengths and thus possesses higher potential in quantifying both genes and isoforms in an unbiased manner.

3GS sequencers provide a unique application interface commonly known as Read-Until (RU). RU had been employed to implement selective sequencing on DNA by ejecting the unwanted reads computationally (Bao, Wadden et al. 2021, Kovaka, Fan et al. 2021, Payne, Holmes et al. 2021). Selective sequencing or targeted sequencing is the sequencing approach to selectively sequence any given subset of DNA or RNA molecules from a pool of molecules. In general, selective sequencing is divided into three steps: (i) Obtaining the electrical signal of the read currently being sequenced. (ii) Determining the origin of the read computationally. (iii) Sending the ejection command to the sequencer via RU along with the ID of unwanted reads. Upon receiving the ejection command, the sequencer can invert the electrical field polarity applied across the designated pore for a period of time to eject the current read, freeing the pore to sequence more reads without any biochemical-based enrichment or additional library preparation step.

Therefore, this technology has the potential to revolutionize the field of in-vitro diagnosis for developing novel clinical applications for pathogen identification, antibiotic resistance testing, genetic testing, and cancer diagnosis with reduced cost and simplified procedures by selective sequencing.

Pathogen identification and surveillance in clinical samples is important for epidemic outbreak detection or antibiotic resistance testing. For example, the cause of suspected infections in hospitalized patients often remains undiagnosed, resulting in delayed or inadequate treatment, prolonged hospital stays, readmissions, and increased patient mortality and morbidity (Friedman, Letai et al. 2015). The number of cases of sepsis in US had increased 71% from 2003 to 2007, increasing the demand for rapid pathogen detection (Lagu, Rothberg et al. 2012). Existing pathogen identification methods such as tissue culturing, or Next-Generation Sequencing (NGS)-based pathogen identification are time-consuming and often require performing technically complex protocols. This delays the identification of pathogens and impairs clinical outcomes. With read length significantly longer than existing NGS-based method (150 bp), pathogen identification can be achieved in nanopore sequencing using fewer reads. With the help of nanopore selective sequencing, DNA/RNA sequences originating from the host (e.g., human patient) are ejected during sequencing without any modification to the library preparation protocols, the turnaround time to collect enough reads for the diagnosis is drastically reduced and thus reducing cost per sample, shortening the diagnosis time.

Copy Number Variation (CNV) is a type of structural variation of the genome, including multiplication and deletions of a particular segment of the genome (>1 kb) (Stratton, Campbell et al. 2009). For example, CNV around the gene FMRI (fragile X mental retardation 1) gene is often related with fragile X syndrome, a developmental disorder that affects cognitive and behavioral functions (Nagamani, Erez et al. 2012). CNV in prostate cancer is associates with increased risk of metastases at diagnosis (Grist, Friedrich et al. 2022). In addition, Single Nucleotide Polymorphisms (SNP) and genome phasing has been an important research field because their association with diseases (Onay, Briollais et al. 2006, Khamlichi and Feil 2018). Due to its shorter read length, existing NGS-based method requires high coverage at the genomic region of interest which drives up the sequencing cost. It may also have difficulties solving the complex genomic regions (Ekblom and Galindo 2011, Nielsen, Paul et al. 2011, Jiang, Wang et al. 2016). The long reads generated by nanopore sequencing can greatly reduce the required sequencing depth required for CNV analysis. With the help of selective sequencing, high coverage around genomic region can be achieved and thus enabling SNP detecting and phasing allele in lower cost and shorter time.

Detecting DNA methylation in cancer-related genes such as 5-methylcytosine (5 mC) and 5-hydroxymethylcytosine (5 hmC) plays an important role in cancer development and prognosis. 5 mC is usually associated with gene silencing, whereas 5 hmC is associated with gene activation (Branco, Ficz et al. 2012). For example, the hypermethylation of tumor suppressor gene RASSF1 (Ras Association Domain Family Protein 1) is frequent in neuroendocrine cancer and prostate cancer. The silencing of RASSF1 gene due to methylation can lead to higher risk of cancer invasion and metastasis (Daniunaite, Jarmalaite et al. 2014, Walter, Rozynek et al. 2018). DNA methylation patterns has been used as biomarkers for treatment response and prognosis, current solutions to investigate the methylation status of cancer-related genes are bisulfite sequencing and Methylated DNA immunoprecipitation Sequencing (MeDIP-Seq) have their drawbacks. For example, MeDIP-seq may introduce biases due to the specificity of the antibody used for immunoprecipitation and provide lower resolutions. While bisulfite sequencing provides methylation at single nucleotide resolution, it requires complex library preparation process such as: bisulfite conversion, adapter ligation, and PCR amplification. The sequencing cost is expensive when a large numbers of samples or large genomic regions need to be investigated. In addition, bisulfite sequencing also having difficulties in distinguishing between 5 mC and 5 hmC (Kit, Nielsen et al. 2012, Cheng, He et al. 2019). In comparison, nanopore sequencing does not require complex library preparation protocol and capable of distinguishing the 5 mC and 5 hmC at single nucleotide resolution by analyzing the electrical signal of captured DNA which is free of the bias from PCR amplification and able to generate consistence result in low coverage. Selective sequencing ensures that only DNA reads from genomic regions of interest are fully sequenced, increasing efficiency and lowering the cost per sample.

The most important advantage of nanopore selective sequencing is that methylation, CNV and phasing analysis can be combined in one sequencing run, further reducing the sequencing cost. During selective sequencing, DNA reads from non-target genomic region are ejected. These ejected reads are partially sequenced and recorded before being ejected. The partially sequenced length is 500˜600 bp while the length of reads from genomic region of interest can reach up to 20 kb, both are longer than related art systems and methods such as NGS, making it possible to conduct CNV analysis after selective sequencing using shallower sequencing depth than NGS method.

### BRIEF SUMMARY OF THE INVENTION

Embodiments of the subject invention provide a flexible integrated platform incorporated with methods such as neural networks for targeted and/or selective sequencing of DNA/RNA reads using 3GS technology such as ONT sequencers which costs about 1,000 USD to startup. Embodiments can be used in a variety of scenarios, including but not limited to rapid and universal pathogen detection for clinical use, depletion of unwanted DNA/RNA for screening test and selective sequencing of disease-related genes (e.g., cancer genes) for precision medicine.

Embodiments of the subject invention provide a universal solution to sequence pathogen-originated sequences selectively and require no modification to the sequencing library preparation protocol nor any prior knowledge on the composition of the sample. Therefore, swift identification of pathogens and better clinical outcomes can be achieved by ejecting host DNA/RNA (e.g. human originated DNA/RNA) during the sequencing of clinical samples.

Another potential application of the invention is the depletion of unwanted DNA/RNA from specific genomic regions which are deemed unnecessary. Ejecting these unwanted DNA/RNA can increase the effective yield of the flowcell when the genomic regions of interest aren't clearly defined during trial experiments. For example, ribosomal RNA (rRNA) constitutes approximately 90% RNA species in total RNA and it is detrimental to the whole transcriptome analysis and thus must be depleted (O'Neil, Glowatz et al. 2013). Despite the effort of polyA⁺ enrichment employed in the nanopore DRS protocol to remove rRNA, mitochondrial RNAs (mt-RNAs) and rRNA still takes up ˜30% of the sequenced read, reducing the effective yield of a MINION flowcell (Mercer, Neph et al. 2011, Mock, Braun et al. 2023). It is because certain mt-RNAs and rRNA cannot be removed by existing protocol (Slomovic, Laufer et al. 2005). Therefore, the effective yield of a flowcell can be increased using selective sequencing by ejecting mt-RNAs and rRNA during sequencing.

In selective sequencing of disease-related genes and cancer diagnosis, embodiments can detect mutations in cancer-related genes and guide treatment decisions. Embodiments of the subject invention provide an integrated solution to investigate expression level of disease-related genes, DNA/RNA modification, and copy number variation using one sequencing library in a single sequencing run. In comparison, related art methods often require separate sequencing libraries to be generated, increasing both the complexity and the cost.

Unlike related art methods (e.g., methods relying on NGS), nanopore sequencing conducts DNA/RNA sequencing by measuring the characteristic electrical signal when DNA/RNA moves across a pore that is only nanometers in diameter, generating DNA/RNA reads (10 k˜50 k bp, up to 100 k bp) which are significantly longer than NGS method (150 bp). The sequencer advantageously allows reads to be ejected during sequencing in real time through a programable interface that opens a new way for achieving the selective sequencing computationally. Compared to related art systems and methods, nanopore sequencing can achieve sufficient coverage at genomic regions of interest or species of interest within a shorter time, increasing yield and reducing costs. To implement this selective sequencing strategy, related art methods require expensive hardware including a graphics processing unit (GPU) with limited accuracy, limited scalability, and higher upfront costs.

Current methods for selective sequencing based on the Next Generation Sequencing (NGS, the predecessor to 3GS) platforms are complicated, they usually require sophisticated probe design and lengthy library preparation procedures (Mamanova, Coffey et al. 2010, Gaudin and Desnues 2018). Different kinds of probes and kits need to be constantly updated and restocked for each specific clinical application. Furthermore, the high cost of one NGS sequencer (e.g., 100 k˜650 k USD) prohibits the wide adoption of the NGS platforms in primary hospitals where budgets are usually limited (Quail, Smith et al. 2012). In comparison, the cost of a nanopore sequencer is at least 100× cheaper than its NGS counterparts (e.g., less than 1,000 USD), making this invention a more economical option. Moreover, this invention requires less than 10 reagents/kits which are commercially available for library preparation and no reagents/kits for selective sequencing, minimizing the cost for stock management and staff training.

Embodiments of the subject invention provide a novel selective sequencing method using the initial section (e.g., only the first few seconds) of the electrical signals directly to decide if the sequenced reads are from selected genomic regions or from different species. Embodiments provide Artificial Intelligence (AI) models established by training deep learning neural networks using collected nanopore sequencing signals. The frontend and backend design allows this invention to be easily integrated into the existing nanopore sequencing infrastructure, offering real-time parallel molecule classification with the flexibility to meet the requirements of a variety of selective sequencing applications such as detecting pathogens in clinical samples, targeted gene panels, and other applications.

### BRIEF DESCRIPTION OF THE DRAWINGS

FIGS. 1A-1C illustrate a system architecture in accordance with an embodiment of the subject invention.

FIGS. 2A-2C illustrate a neural network architecture in accordance with an embodiment of the subject invention.

FIGS. 3A-3C illustrate a library preparation workflow of nanopore direct RNA sequencing protocol (FIG. 3A) with an example electrical signal of one sequenced RNA (FIG. 3B) and histogram of the poly(A) tail length of all reads which passed quality control (FIG. 3C) in accordance with an embodiment of the subject invention.

FIGS. 4A-4E illustrate an example of a Pruned Exact Linear Time (PELT) method for adapter and poly(A) tail segmentation. FIG. 4A represents the original signal, while FIGS. 4B-4D each, respectively, represents the segmented signal with different number of break points whose locations marked with green vertical short-dashed lines and the mean of each segmented region illustrated by a red horizontal long-dashed line in accordance with an embodiment of the subject invention. FIG. 4E is the histogram which illustrates the high correlation between the PELT method and related art nanopolish, a poly(A) segmentation method designed for offline analysis. The X axis denotes the time differences in poly(A) length of the same read estimated by PELT and related art. The Y axis is the count of each bin in the histogram (bin width: 0.2 s). The mean value of the time differences between PELT and related art is 0.20 s, standard deviation is 0.64 s.

FIG. 5 illustrates the classification accuracy comparison of various approaches in accordance with an embodiment of the subject invention. After hyperparameter tunning using Bayesian optimization (BO), the accuracy of the LSTM neural network can be increased by 5.49% and surpasses existing methods.

FIGS. 6A-6E illustrate the pathogen genome coverage comparison between control and experiment groups with respect to time in accordance with an embodiment of the subject invention.

FIGS. 7A-7C illustrate the showing genome coverage comparison between target genomic region and non-target genomic region with respect to time in accordance with an embodiment of the subject invention.

FIG. 8 illustrates a workflow for real-time selective nanopore sequencing in accordance with an embodiment of the subject invention.

FIGS. 9A-9E illustrate performance comparisons between serial vs. parallel processing and GPU vs CPU (host) in accordance with an embodiment of the subject invention.

FIGS. 10A-10E illustrate confusion matrices of the neural network trained with various signal length in accordance with an embodiment of the subject invention.

FIGS. 11A-11C illustrate Copy Number Variation (CNV) analysis differences between standard nanopore DNA sequencing without selective sequencing (FIG. 11A) and selective sequencing in accordance with an embodiment of the subject invention (FIG. 11B) showing that it is feasible to conduct CNV analysis on top of selective sequencing. FIG. 11C illustrates comparison of the CNV results of the public source (upper figure) and the results of after selective sequencing (lower figure).

FIGS. 12A-12G illustrate DNA methylation analysis differences between LNCAP (prostate cancer cell line) and RWPE1 (normal prostate cell line) on a genomic region of ˜12.3 kb in length, containing 336 CpG sites, according to an embodiment of the subject invention. FIG. 12A illustrates the median Log Likelihood Ratio (LLR) of all the 336 CpG sites in a heatmap, the higher LLR the more likely this CpG site is methylated. FIG. 12B illustrates the threshold for determining the status (methylated, unmethylated, ambiguous) of each respective site. FIG. 12C illustrates selected statistics of differently methylated CpG islands identified between LNCAP and RWPE1.FIGS. 12D-12G illustrate the results of Gene Ontology (GO) analysis performed on differentially methylated CpG sites between LNCAP and RWPE1 cells. Specifically, FIG. 12D presents the KEGG pathway analysis results; FIG. 12E depicts the results for Molecular Function (MF); FIG. 12F depicts the Biological Process (BP) results; and FIG. 12G depicts the Cellular Component (CC) results.

FIGS. 13A-13B illustrate the neural network hyperparameter tunning result using Bayesian Optimization (BO, solid line) and Grid Search (GS, dashed line). FIG. 13A illustrates the validation accuracy of the neural network throughout the hyperparameter tunning processes, the higher the validation accuracy, the better the neural network performs when processing unseen data. FIG. 13B illustrates the validation accuracy of the neural network throughout the hyperparameter tunning process, the lower the validation loss, the better.

### DETAILED DISCLOSURE OF THE INVENTION

Embodiments of the subject invention offer numerous advantages and improvements over existing methods, including but not limited to (i) high efficiency and high yield, and (ii) reduced or eliminated upfront cost with high flexibility.

To achieve benefits including high efficiency and high yield, compared to related art, certain provided methods convert the electrical signals to bases (e.g., base calling) and align them to the reference genome to decide if the sequenced read falls into the targeted regions or not. Certain embodiments determine if the sequenced read is on target or not using the electrical signal directly by analyzing its signal features, skipping both base calling and alignment which are computationally extensive. Therefore, embodiments require much fewer computing resources and can be run on lower cost (e.g., desktop, mobile or edge) computers without the need, or with reduced need, for a GPU or other more powerful processor. In addition, embodiments also require shorter reads to do the classification for the non-target reads to be rejected earlier than related art methods, hence improving the final sequencing yield and widening the potential applications. When measured in time, certain embodiments require about 0.5 second of the signal (e.g., equal to about 175 bp) while related art systems require about 3 seconds (e.g., equal to about 1350 bp). Since embodiments of the subject invention advantageously perform with shorter signal/reads, embodiments have lower requirements and hence broader potential application.

To achieve reduced or eliminated upfront cost with high flexibility, the architecture of certain embodiments allows signal classification to be run in remote servers or in the cloud when provided as Software as a Service (SaaS) delivered to researchers who conduct small-scale sequencing in the lab or in the field. Embodiments can also be applied (e.g., licensed) to institutional users who conduct sequencing in parallel and own powerful computing clusters. A variety of AI classification models can be tailored to meet the different clinical or research requirements without any modifications (alternatively, with minimal or reduced modifications) on the client side. In addition, these embodiments also minimize the risk of malicious clients gaining the key AI classification models as the backend is not installed on the client computer.

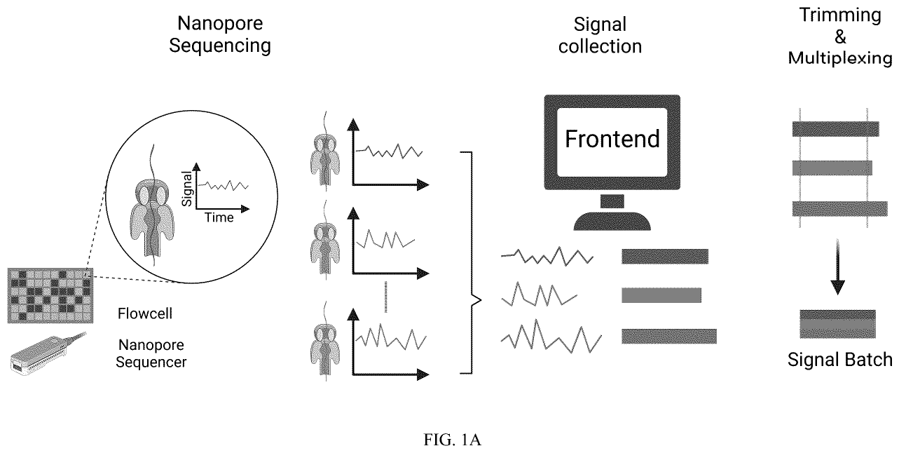

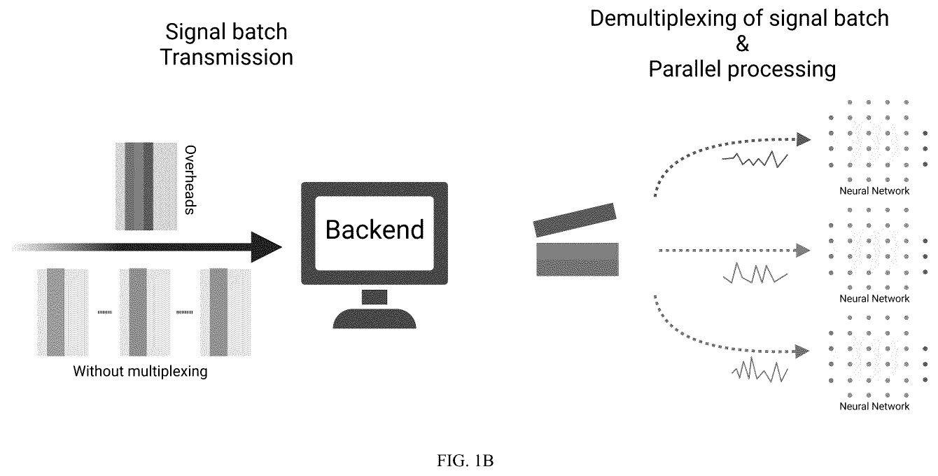

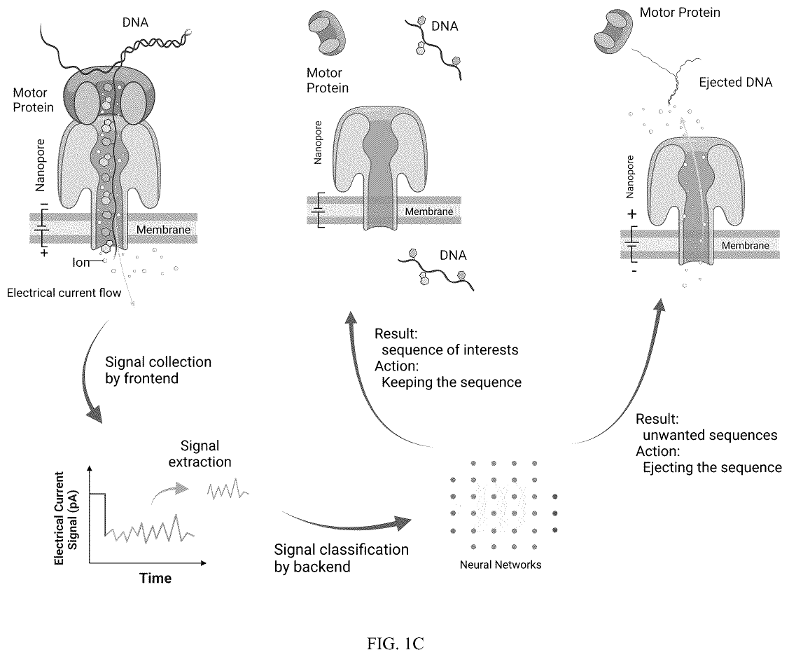

Turning now to the figures, FIGS. 1A-1C illustrate a system architecture in accordance with an embodiment of the subject invention. The architecture of certain embodiments is divided into a frontend and a backend. In certain embodiments the frontend and backend can be run in the same computer/server in the form of local or private network. In certain embodiments the frontend and backend can be run in different computers/servers on a secure local or private network. In certain embodiments the frontend and backend can be run in different computers/servers connected via a virtual private network. In certain embodiments the frontend and backend can be run in different computers/servers connected via a public internet segment. In certain embodiments the frontend can run on a local server in a laboratory or health care facility and the backend can be run as a cloud based or server-based SaaS application or service.

As shown in FIG. 1A, the frontend is a program which communicates with the sequencer controlling software (e.g., MINKNOW) for obtaining nanopore electrical signals and ejecting unwanted reads. In detail, after the sequencing is initiated by the user or the frontend, frontend can constantly (alternatively, repeatedly, iteratively, on a schedule, as needed, programmatically, for a period of time, at intervals, or intermittently) pull the latest signal from sequencer (e.g., via MINKNOW.) Once the length of collected signal exceeds the length requirement for classification, the excess portion of the signal can be trimmed, and the trimmed signal forwarded to the backend for classification. In certain embodiments where multiple signals are collected from MINKNOW, a signal batch can be constructed by trimming, multiplexing and compressing the trimmed signals. FIG. 1B illustrates one embodiment where the signal batch helps reduce transmission overhead and processing delays while saving network bandwidth. In certain embodiments the backend is a program which listens to a specific network port or other data source for incoming multiplexed signal batch data sent by the frontend. After signal batch demultiplexing, the signals can be classified using trained neural networks or other algorithms. In certain embodiments, a provided Long Short-Term Memory (LSTM) based neural network is constructed and trained to identify the origin of a read using the nanopore raw electrical signal. The results are returned to the frontend (e.g., through the same network port, or through a different network port) for ejecting unwanted reads (not illustrated.) FIG. 1C illustrates the detail of nanopore behavior based on the prediction result of the network using DNA as an example, but also applicable to RNA, according to certain embodiments of the subject invention. During sequencing an electrical field is applied across the nanopore and membrane which drives the DNA through the nanopore, after signal processing as mentioned above, the nanopore can have two different behaviors. For any sequence classified as a sequence of interest, the frontend can instruct the sequencer to keep it and allow the DNA to be fully sequenced. FIG. 1C illustrates the sequence of one strand of the DNA, but in certain embodiments, both strands can be sequenced (e.g., using different sequencing library preparation protocols, or alternatively, using the same sequencing library preparation protocol.) For sequences classified as an unwanted sequence, the frontend can instruct the sequencer to reverse the electrical field across the nanopore and membrane and thus eject the DNA which is partially sequenced. By this method, the nanopore is freed to capture new sequences which can originate from genomic regions of interest or targeted species while minimizing or reducing time sequencing the unwanted sequences.

In certain embodiments, an advantageous and novel feature of this architecture is that this enables higher flexibility by offloading the signal classification task to the backend server. By using this configuration, embodiments can be delivered to users with minimal requirements so that the sequencing service can be provided as Software as a Service (SaaS) or licensed to the users. These provided elements allow embodiments to be integrated into existing infrastructure easily, facilitating clinical and commercial adoption.

One advantageous and novel feature of this architecture is live signal trimming, depending on the sequencing library used, auxiliary sequences such as adapter sequences, poly(A) sequences, etc. are ligated to the beginning of the DNA/RNA molecules and appear in the beginning of the signal. Though auxiliary sequences are an important part of the library preparation procedures, their signals can be detrimental to the performance of the signal classification as it carries useless RNA sequences. Related art systems remove the adapter sequences during the computationally expensive base calling process which converts the electrical signal to DNA/RNA sequences using GPU. By exploiting the characteristic of the adapter sequences, embodiments of the subject invention can accurately trim the corresponding signal of the auxiliary sequences in real-time with increased efficiency.

Another advantageous and novel feature of this architecture is that this reduces the transmission overhead when transmitting the signals to the backend. Without signal multiplexing, transmission overhead and latency accumulates as each signal is sent to the backend. In comparison, signal multiplexing greatly reduces the overhead and the latency as well as enables parallel processing in the backend which improves the efficiency of signal processing and reduces the processing latency.

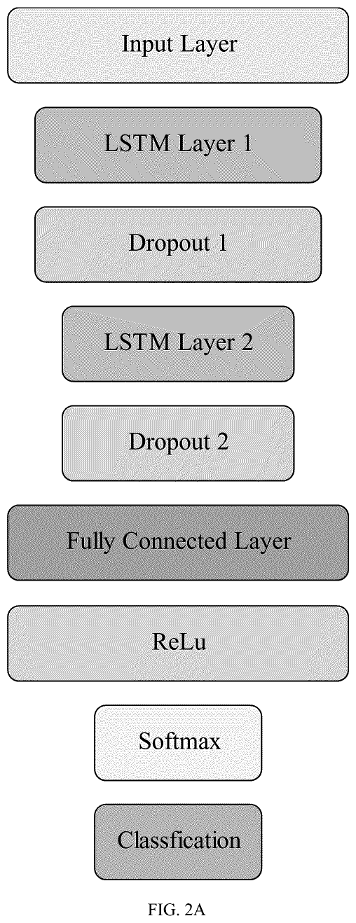

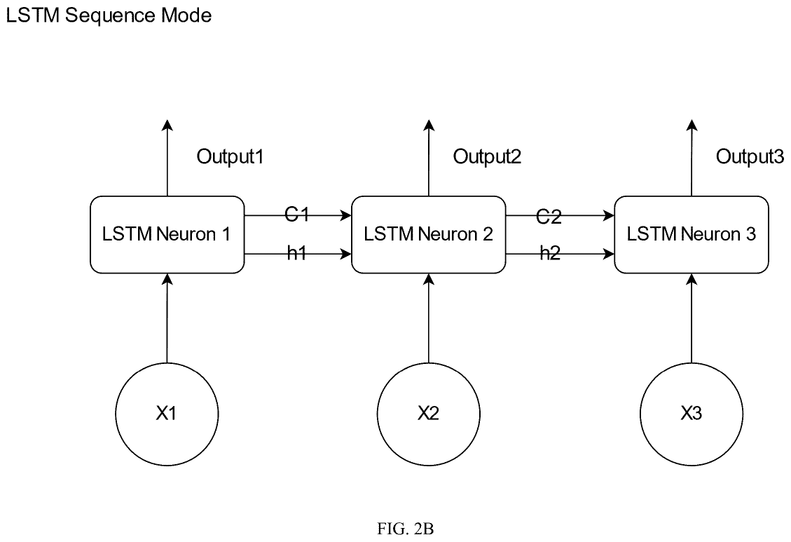

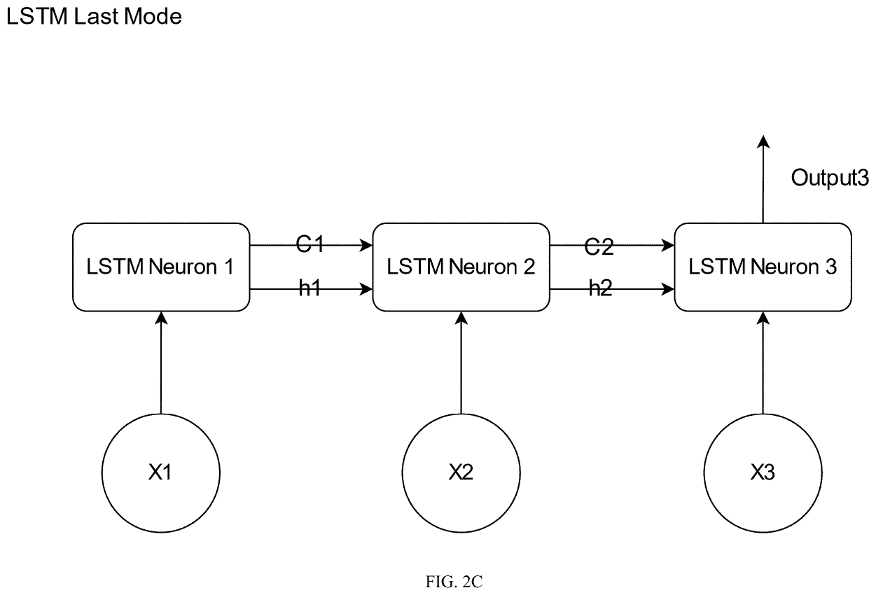

FIG. 2A Illustrates a neural network architecture in accordance with an embodiment of the subject invention. Each layer is color coded with the same type of layer sharing the same color for case of visual identification.

The first layer of the network is the input layer which size is influenced by the length of input signal. In certain embodiments, additional features are added to facilitate prediction. (e.g., the frequency density of the signal).

The second layer (LSTM Layer 1) is the Long Short Term Memory (LSTM) layer with 120 LSTM neurons which extract and abstract useful information from the input layer.

The third layer (Dropout 1) is the dropout layer which randomly disables some of the LSTM neurons with probability of 0.25 during neural network training. (e.g., disabling 25% of the LSTM neurons in the second layer) Dropout layer increases the robustness of the neural network, inhibiting it from remembering the answer of each input rather than learning the general patterns during neural network training. A dropout layer does not participate in any calculation during training and is disabled afterwards.

In certain embodiments, the number of LSTM neurons can be reduced; the dropout probability of the dropout layer is adjustable between 0 and 1; it is also contemplated to repeat the second and third layer when the prediction performance is low.

The fourth and fifth layers (LSTM Layer 2 & Dropout 2) serve the same purpose as the second and third layer. However, in certain embodiments, the fourth & fifth layer can be omitted and the mode of second layer (LSTM Layer 1) switched to Last Mode in embodiments where satisfying prediction performance (e.g., accuracy >80%) is achieved using only the second and third layer.

The sixth layer (Fully Connected, or FC Layer) serves as the bridge which converts the highly abstracted pattern extracted in LSTM layers to the final prediction result. As the name Fully Connected (FC) layer suggests, each neuron in FC layer receives input from every other neuron in the preceding LSTM layer. The number of outputs is always determined by the number of prediction labels during neural network training. In certain embodiments, the number of outputs is equal to two, indicating whether the signal is coming from genomic region of interests or specie of interests.

The seventh layer (ReLu) is employed to improve the overall prediction performance of the network. It converts all negative values in the input to zero, and all positive values remain unchanged. Due to its simplicity, computation time can be reduced compared to other alternatives such as tanh or sigmoid.

In the end, the eighth and ninth layers (Softmax and Classification, respectively) output the prediction result for the input signal. Similar to FC layer, the number of outputs is also determined by the number of prediction classes during neural network training. For each prediction label, the Softmax layer assigns a probability and the sum of all probability is always equal to 1. Classification layer can select the prediction label with highest probability as the prediction result.

FIGS. 2B-2C illustrate the differences between Sequence Mode (SM) and Last Mode (LM) of LSTM layer in certain embodiments of the subject invention. As the name suggested, when configured to SM, each neuron outputs to the next layer of the neural network in sequence; when configured to LM, only the last neuron outputs to the LSTM layer. Each neuron in the LSTM takes three inputs: (i) the output from previous layer. (ii) cell state C which contains long term memory from the last neuron of the current LSTM layer, (iii) hidden state H which contains short term memory from the last neuron of the current LSTM layer. Similarly, each neuron in the LSTM layer has three outputs: (i) the output to the next layer. (ii) cell state C which outputs long term memory to the next neuron of the current LSTM layer, (iii) hidden state H which outputs short term memory to the next neuron of the current LSTM layer. Important information extracted from the signal is stored in the three outputs and passed on to the next LSTM neuron or next layer. For any given input signal, only information deemed important is extracted and passed on as it fed through the LSTM layer. The output of the last neuron in a LSTM layer contains the concentrated information which takes full consideration of the input. Therefore, LM is generally used in the last LSTM layer where all the processing is finished, while SM is generally used in other situations.

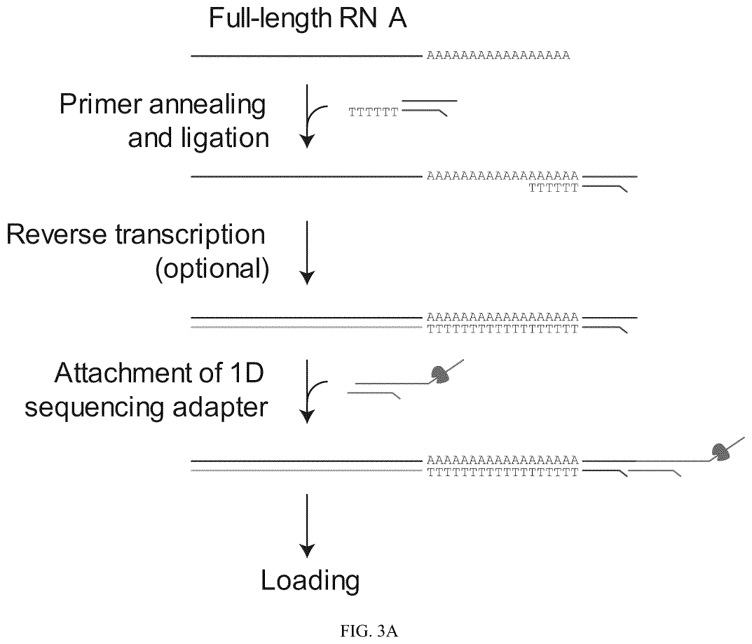

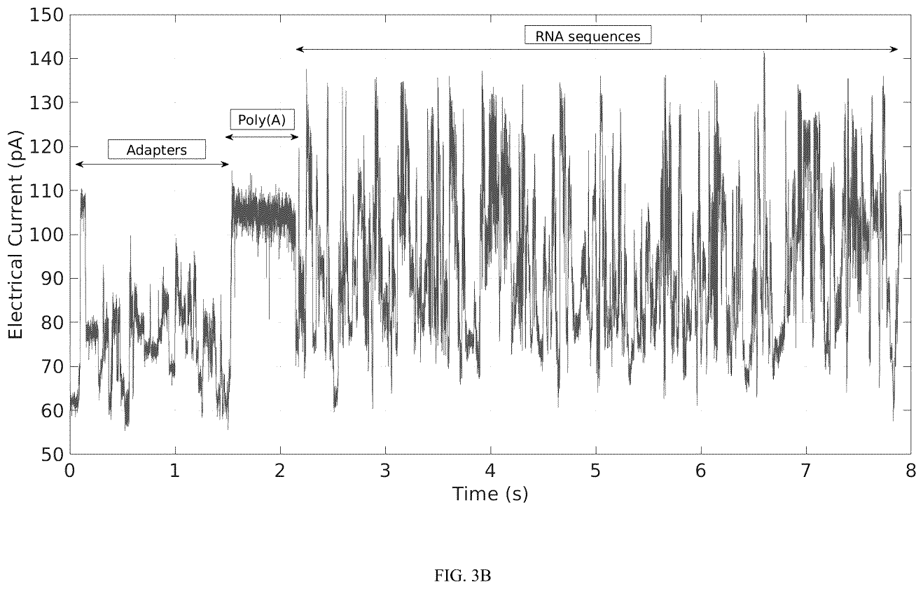

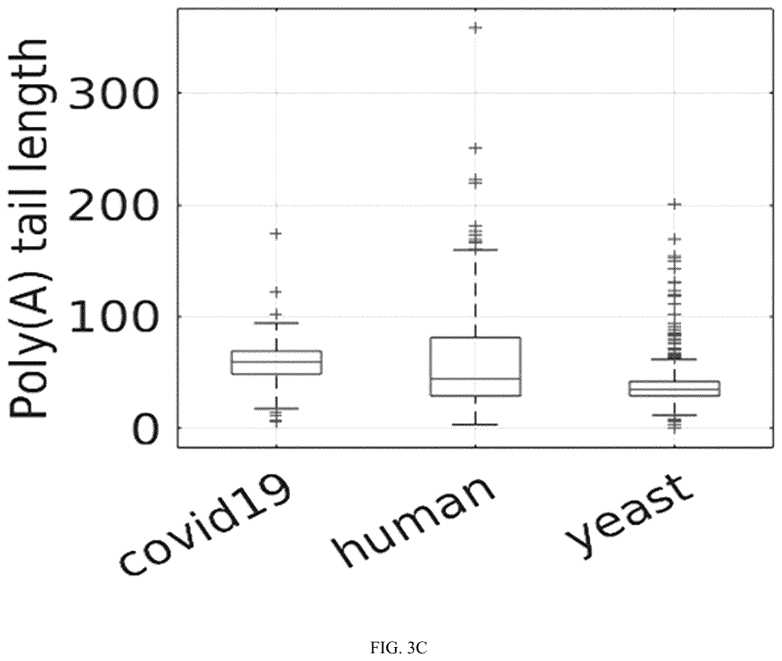

FIGS. 3A-3C illustrate a library preparation workflow (FIG. 3A) of nanopore direct RNA sequencing (dRNASeq) protocol with an example electrical signal (FIG. 3B) of one sequenced RNA and boxplot (FIG. 3C) of the poly(A) tail length by species which passed quality control in accordance with an embodiment of the subject invention.

FIG. 3A illustrates that one side of the full-length RNA contains a section made of multiple adenosine monophosphates which is known as the poly(A) tail. After the library preparation, only prepared RNA with a poly(A) tail will be sequenced, starting from the direction of the adapter. Therefore, as illustrated by FIG. 3B, there should be an adapter and a poly(A) tail at the beginning of each signal of sequenced RNA. However, as illustrated by FIG. 3C, the length of the poly(A) tail can vary. Each of the adapter and poly(A) tail, respectively, are detrimental to determine the origin of the sequence reads because each contains little sequence information and thus is advantageously trimmed before the prediction by certain embodiments of the subject invention. The detection and trimming of both adapters and poly(A) tails during the real time nanopore selective sequencing is a uniquely novel and advantageous feature of certain embodiments of the subject invention.

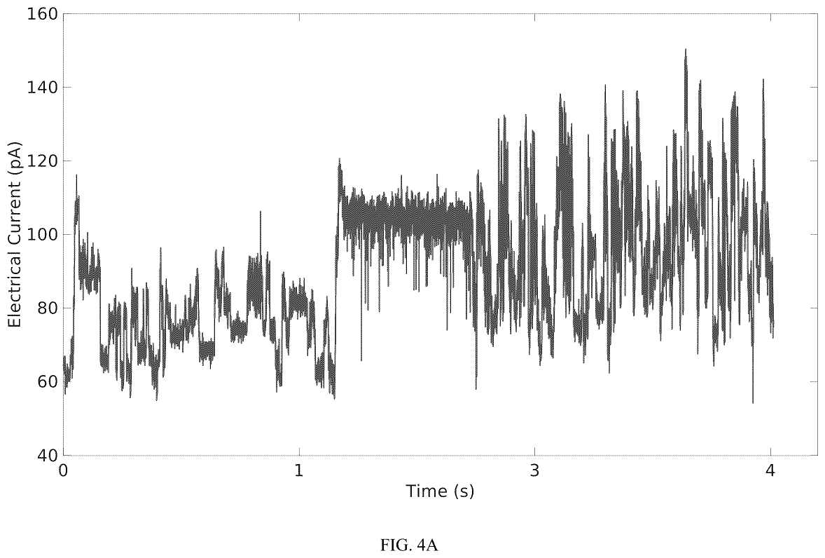

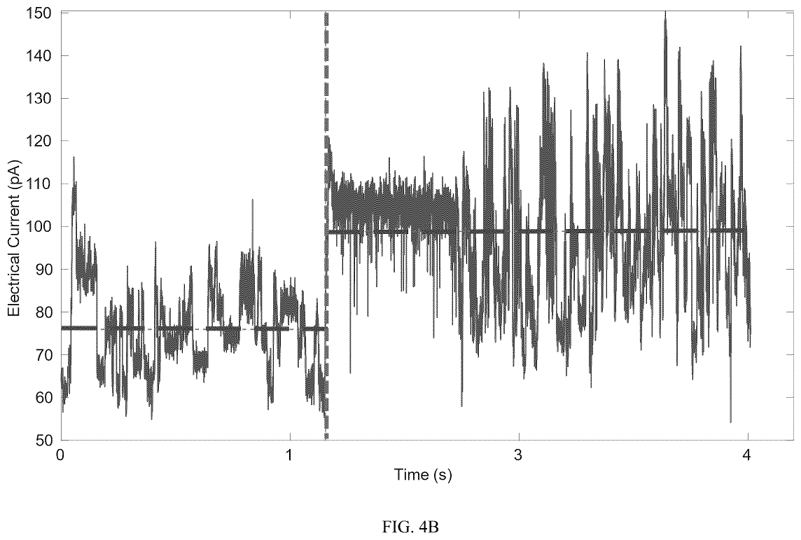

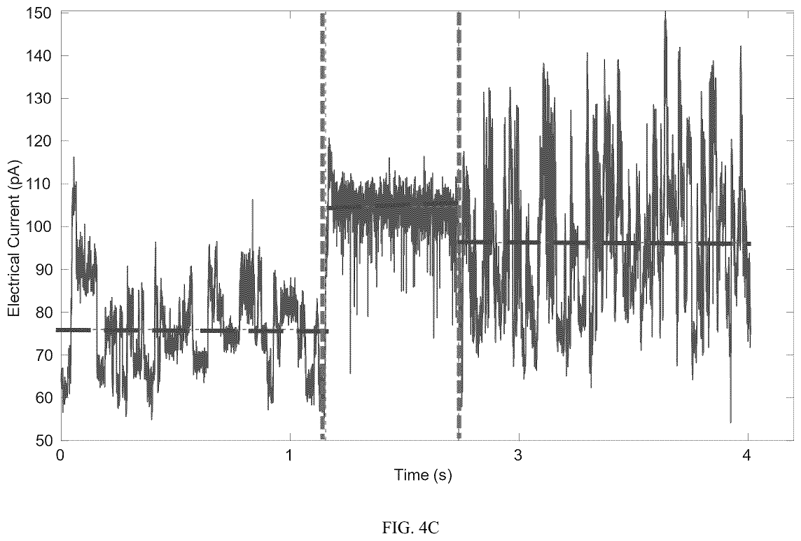

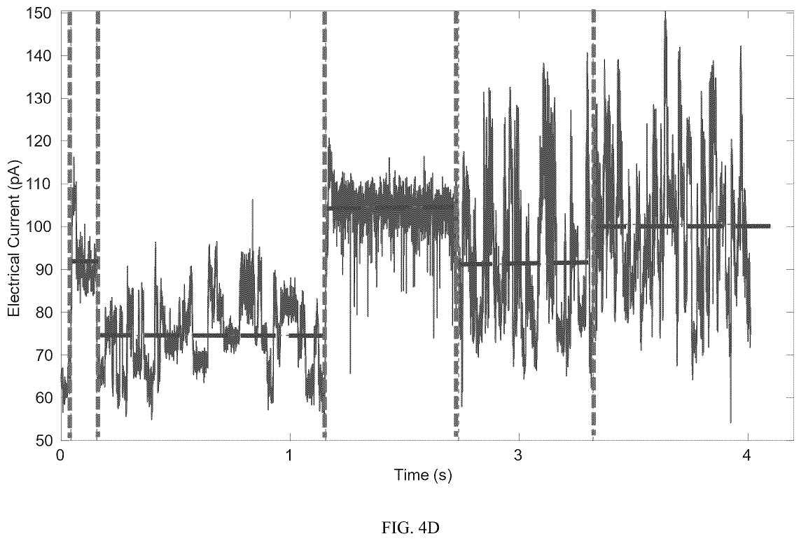

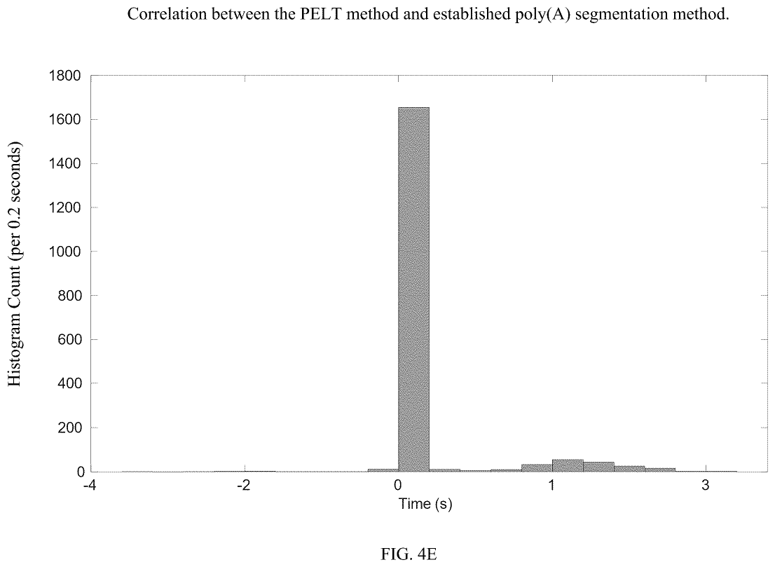

FIGS. 4A-4E illustrate an example of a Pruned Exact Linear Time (PELT) method for nanopore electrical signal segmentation and trimming.

FIG. 4A represents the original signal, while FIGS. 4B-4D each, respectively, represents a segmented signal with the locations of break points marked with vertical lines and the mean of each region illustrated by a horizontal line in accordance with an embodiment of the subject invention.

In detail, the poly(A) has two key characteristics which can be advantageously leveraged to separate it from the adapter and RNA sequences: (i) the abrupt transition in electrical signal between the poly(A) and adapter; and (ii) Poly(A) tail is relatively statistically stable compared to adapter and RNA sequences. To take advantage of the two characteristics, the PELT algorithm iteratively finds N change points where the signal had changed drastically so that the signal is separated into N+1 regions where the residual squared error between the signal in the region and the localized mean is minimized. In certain embodiments, the N is set to 2 so that the signal is segmented into 3 sections corresponding to adapter, poly(A), and RNA sequences but can be adjusted according to the actual situation. As illustrated by FIG. 4B, the location of first change point is marked by a vertical dashed line and the respective mean of each of the two region it separates are marked by respective horizontal dashed lines. As illustrated by FIG. 4C, on top of the first change point, the second change point is identified and marked with vertical dashed line and the respective mean of each of the three regions the two change points separate are marked by respective horizontal dashed lines where the location of two change points separates the adapter, poly(A), and RNA sequences with a high degree of precision. FIG. 4D illustrates the signal segmentation result when N is set to 5, demonstrating that the signal can be segmented into finer sections as the PELT algorithm iterates.

FIG. 4E illustrates the high correlation between the PELT method and established poly(A) segmentation method designed for offline analysis such as nanopolish. The X axis denotes the time differences in poly(A) length of the same read estimated by PELT and related art nanopolish. The Y axis is the count of each bin in the histogram (e.g., bin width: 0.2s ). The average time difference between the change points identified by PELT and the established method is 0.20 seconds while the standard deviation is 0.64 seconds; suggesting similar performance between the two methods. However, PELT algorithm is more efficient and more suitable for nanopore selective sequencing than the established related art method. For example, the established method segments the signal into four regions using Hidden Markov Model (HMM) to estimate the length of poly(A) tail region for post-sequencing analysis. Therefore, it requires four input files as follows: (i) FAST5 file containing the complete signal of the RNA; (ii) FASTQ file containing the RNA sequences which is extracted from FAST5 file; (iii) BAM file containing the alignment result of the RNA; and (iv) indexing file built using FAST5 & FASTQ files. The FAST5 file are only generated once the DNA/RNA is completely sequenced, and the remaining input files are usually generated after extensive calculation. In comparison, the PELT algorithm requires only the signal collected from the sequencer and segmentation is finished in real time with minimal CPU demand while the related art methods are not suitable for real-time nanopore selective sequencing.

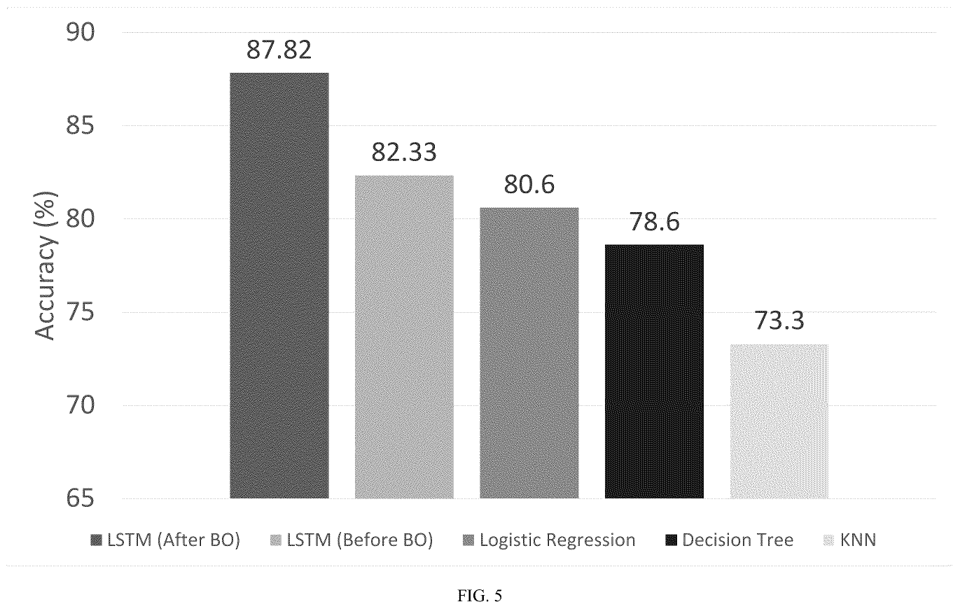

FIG. 5 Is a chart showing a classification accuracy of various approaches in accordance with an embodiment of the subject invention. LSTM outperforms other algorithms such as logistic regression, decision tree, and KNN with respect to accuracy using 4 seconds of the signal for nanopore dRNASeq selective sequencing. The accuracy is calculated by firstly asking the algorithms to predict a group of validation datasets, then summing up the number of prediction results which match the ground truth label in the validation dataset and dividing the sum by the size of validation dataset. Most importantly, by tunning the hyperparameters of the LSTM neural network (e.g., batch size, learning rate) using a Bayesian Optimization (BO) method, the accuracy increases from 82.33% to 87.82%.

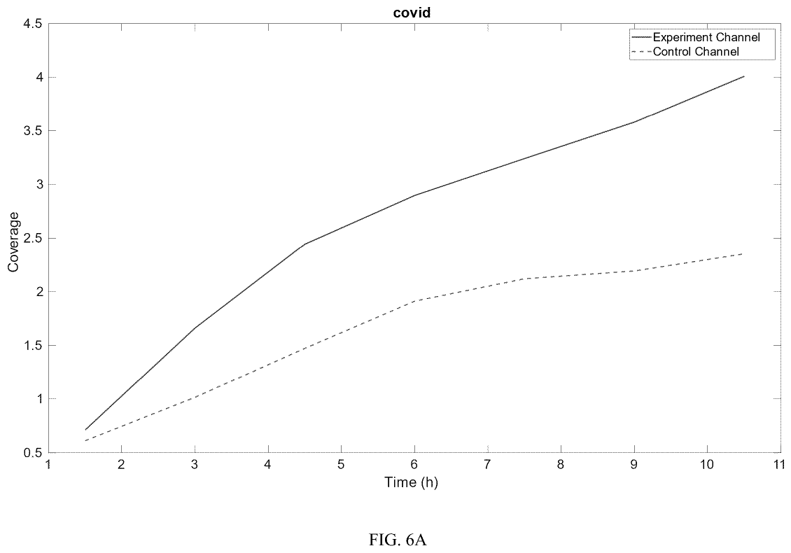

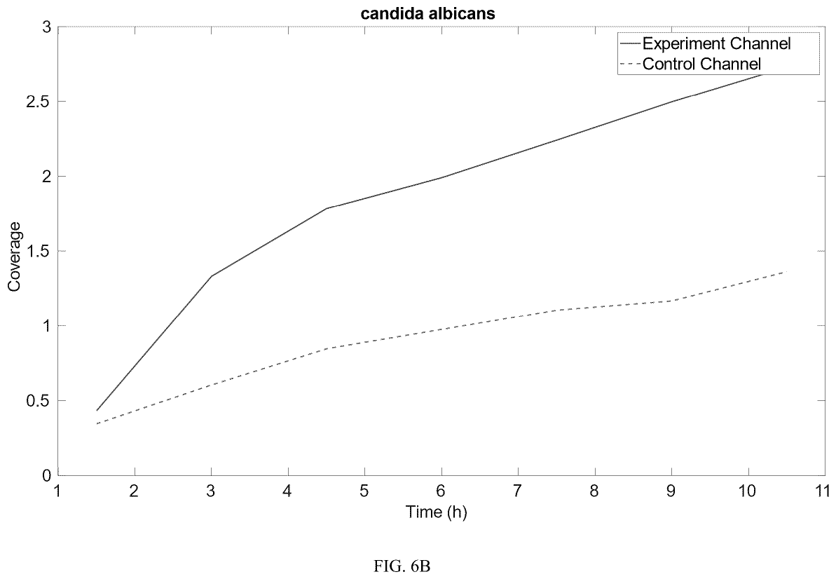

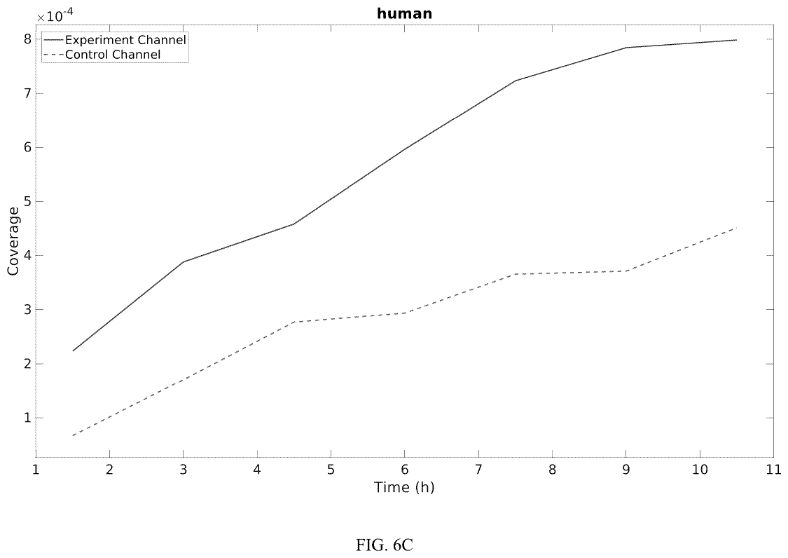

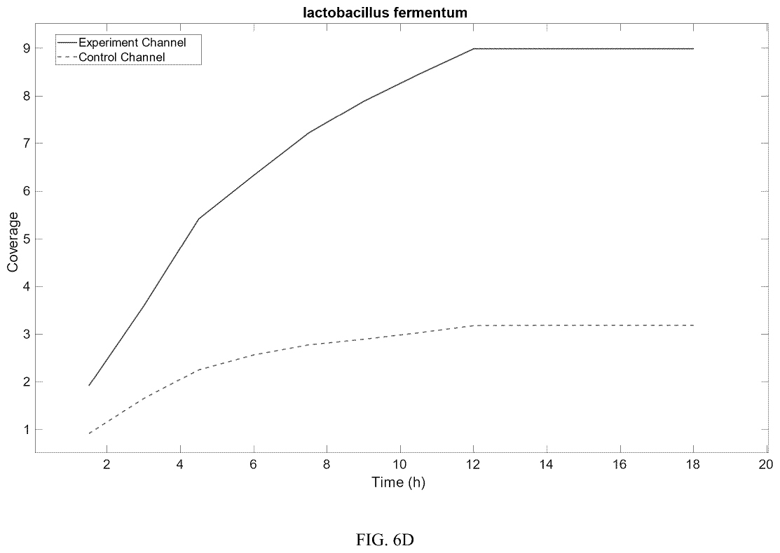

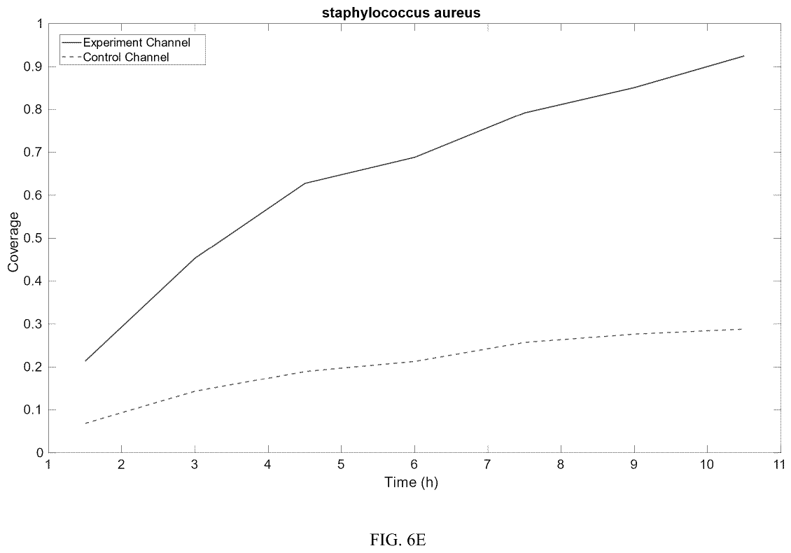

FIGS. 6A-6E illustrate plots showing the genome coverage changes over time by species between control (dashed line) and experiment groups (solid line) in accordance with an embodiment of the subject invention. In detail, one flow cell is programmatically split into two groups: control group and experiment group, selective sequencing is enabled only for the experiment group while the control group conducts normal sequencing. This allows the two groups to be subject to the same sequencing library and compares their yield with minimal bias. As illustrated, selective sequencing can significantly increase the yield of pathogen-originated sequences which can be translated into shorter turnaround time for the clinical diagnosis.

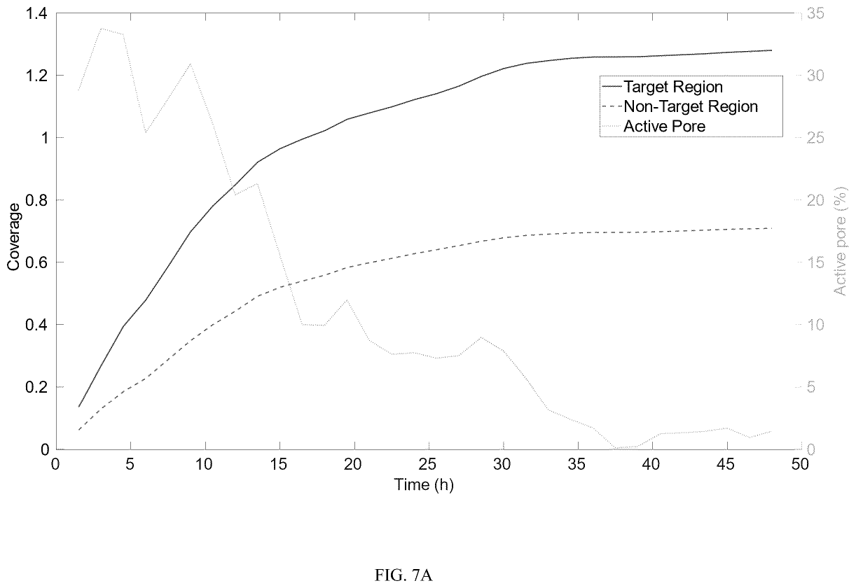

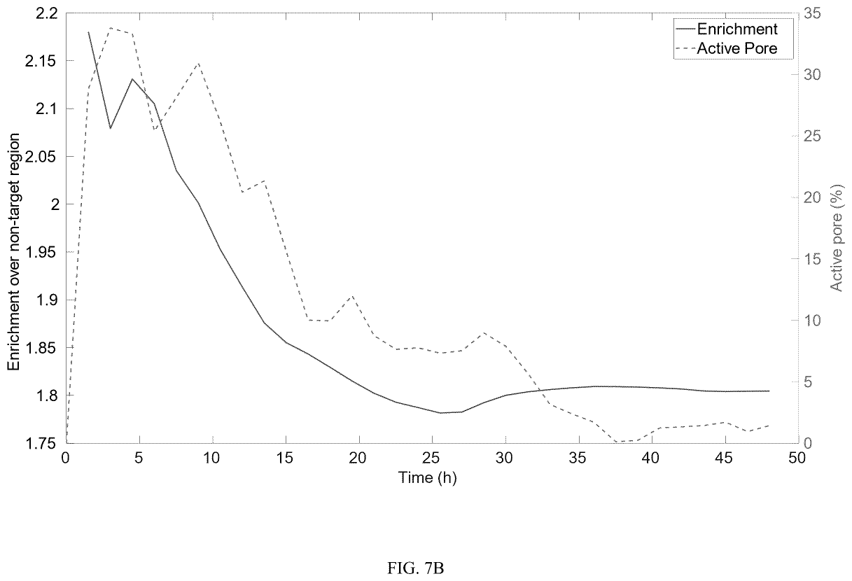

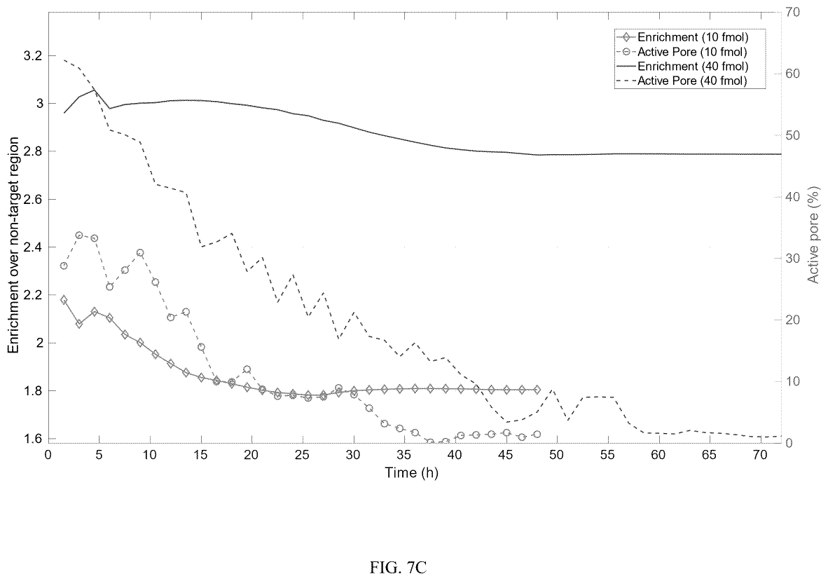

FIGS. 7A-7C illustrates that the enrichment performance of selective sequencing is influenced by percentage of active pores in the flowcell and concentration of the library. These figures are generated during DNA selective sequencing on specific genomic region of the human genome which size is about 10% of the whole human genome. The concentrations of the library are 10 fmol and 40 fmol, respectively, both are within the recommended range for standard nanopore sequencing, each library is sequenced with the same type of flowcell.

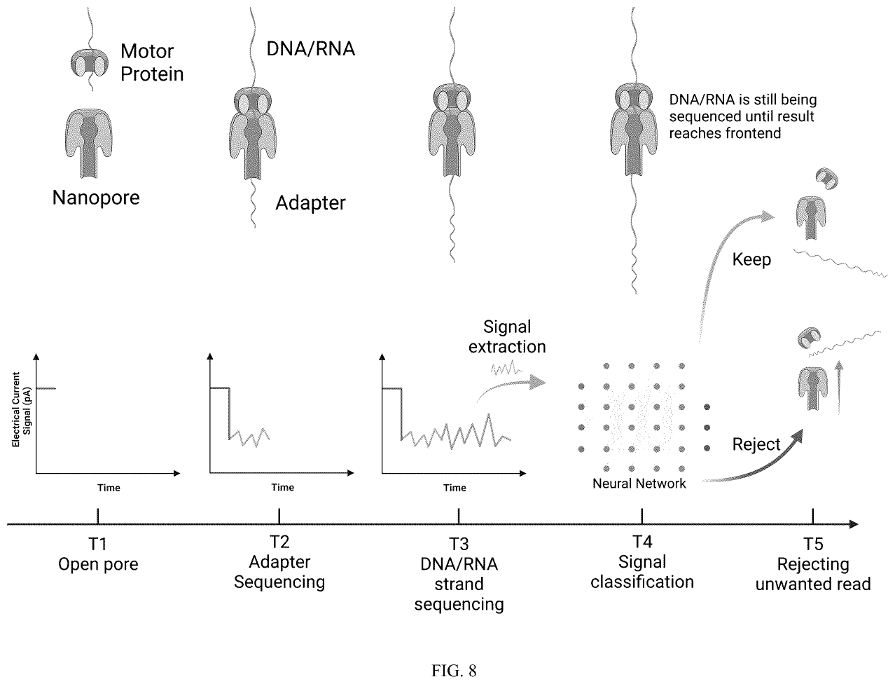

FIG. 8 illustrates a 5 stages workflow for real-time nanopore selective sequencing separated in accordance with an embodiment of the subject invention. At stage T1, the pore is in open state, the electrical current flow through the nanopore is high and stable. At stage T2, the motor protein binds to the nanopore and begins the sequencing with adapters being sequenced first. (Other auxiliary sequences such as poly(A) are not shown in this example.) Driven by the electrical field, the DNA/RNA strand moves across the nanopore and partially block the nanopore. Hence, there will be fluctuation in the electrical signal which contains information about the current DNA/RNA read as well as its modifications. Stage T3-T5 are the most challenging stages of real-time selective sequencing as the duration of these stages should be minimized. During these stages, the DNA/RNA is still being sequenced until the result reaches the frontend. The longer it takes, the longer the DNA/RNA will be sequenced and the lower the performance of selective sequencing will be. Related art systems and methods have several limitations: (i) the approach of related art systems requires powerful computational hardware such as a GPU, that will drive up the upfront cost; (ii) to minimize the duration of stage T3-T5, related art systems usually employ a fast mode during selective sequencing which trades accuracy for speed; and (iii) the required length is too high to be employed in clinical settings. For example, the median length of the RNA sequenced using nanopore dRNASeq protocol is 1,191 nt (length in nucleotides) while the optimal input length for related art systems is about 1,200 nt. This means that related art systems must give suboptimal results for the majority of the reads. In comparison, embodiments of the subject invention require significantly shorter input length: ˜350 nt for DNA, ˜280 nt for RNA (including adapters sequences & poly(A) tail) with the lowest computational resources requirement while providing remote processing functionality.

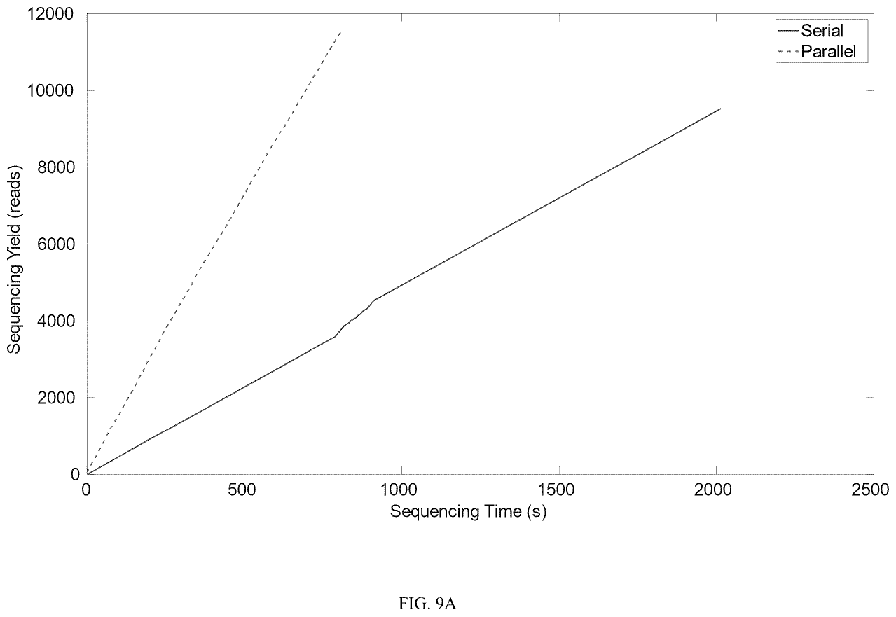

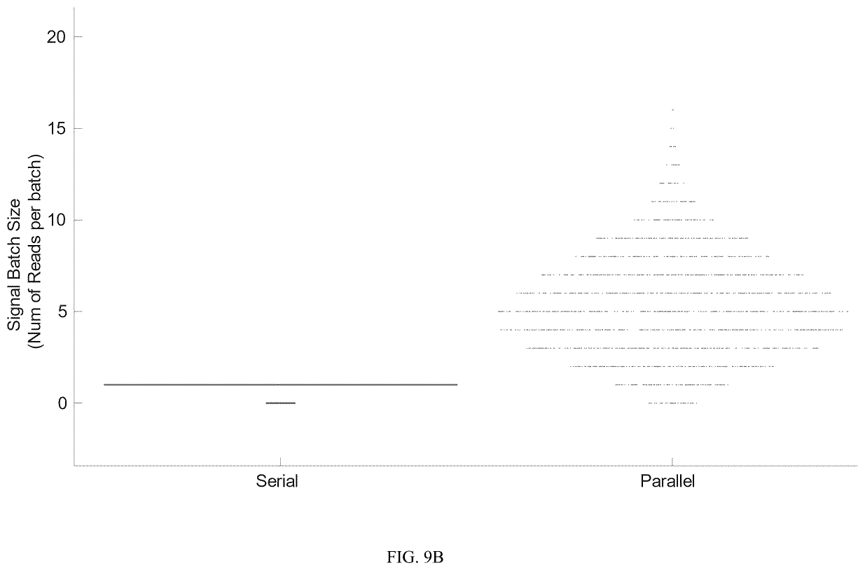

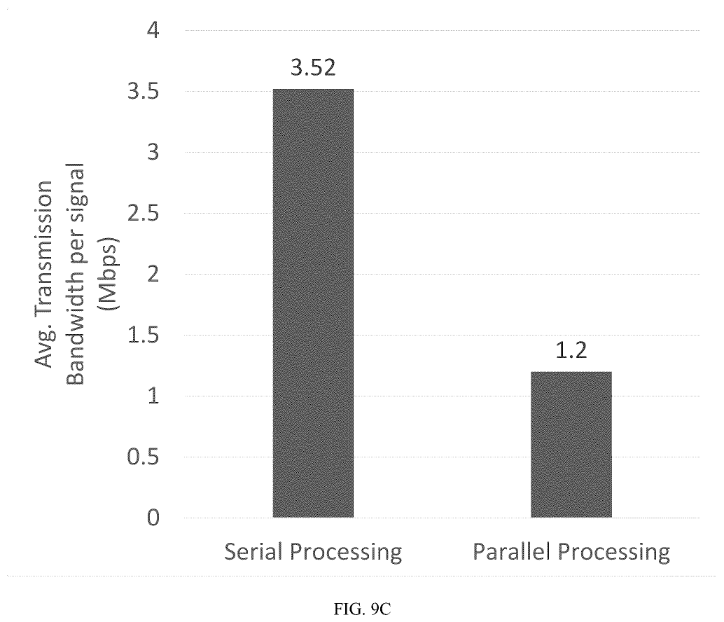

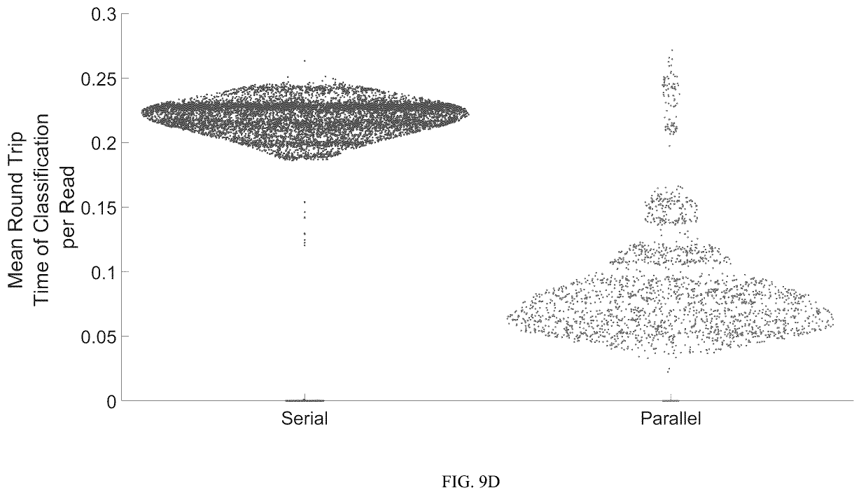

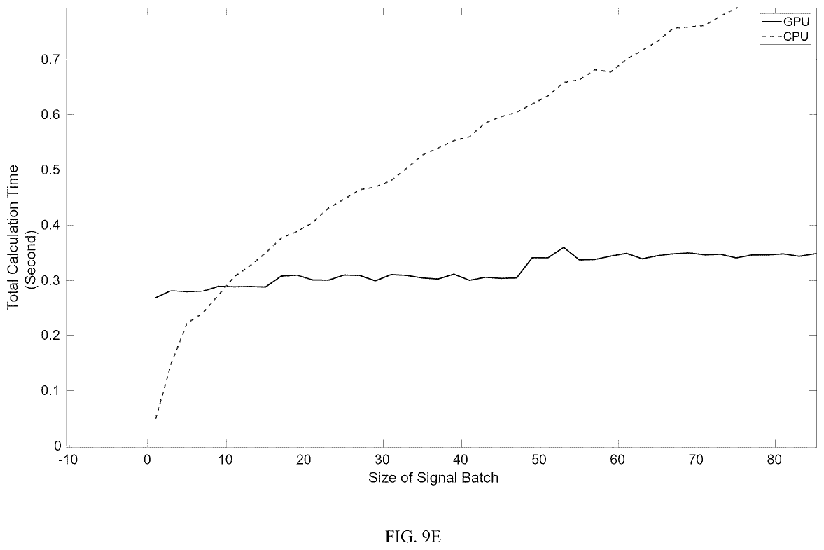

FIGS. 9A-9E illustrate performance comparisons between serial vs. parallel processing and GPU vs CPU (single thread) in accordance with an embodiment of the subject invention.

FIG. 9A illustrates the number of processed reads during two simulation runs of the recorded nanopore sequencing via playback. The yield of parallel processing (dashed line) is 3.71 times higher than serial processing (solid line) after 500 seconds of sequencing. The only difference between playback and actual sequencing is that the playback randomly selects the electrical signal of recorded reads and forwards it to the frontend of an embodiment of the subject invention. This allows for a more controlled environment for comparing the performance between serial vs. parallel processing and GPU vs CPU.

FIG. 9B illustrates the differences in signal batch size between serial processing and parallel processing. For serial processing, only one signal is sent to the backend for processing at a time, transmission and processing delays are added with each transmission. In comparison, the parallel processing allows multiple signals to be transmitted in one signal batch which greatly reduces transmission bandwidth.

FIG. 9C illustrates the differences in transmission bandwidth between serial processing and parallel processing. Parallel processing requires 2.98 times less transmission bandwidth per signal than serial processing. This is important for embodiments of the subject invention to transmit the signal (e.g., over a public internet segment, virtual private network, or private network) with maximal efficiency.

FIG. 9D compares the mean round-trip time of classification per read between the serial processing and parallel processing. The average classification time per read for parallel processing is about 0.16 shorter than the average classification time per read for serial processing. As multiple signals are collected for the majority of the time during sequencing, employing parallel processing in certain embodiments can offer distinct advantages. This approach can enhance the overall efficiency compared to serial processing, making it more suitable for real-time selective sequencing.

In addition, parallel processing can self-adapt to the yield of the sequencer while serial processing does not. In certain embodiments, by default, the nanopore sequencer automatically checks the status of the pores on the flowcell every 1.5 hours and shuts down the pores in poor condition to maximize yield. The check temporarily holds the sequencing and reduces the yield to almost zero during this time.

FIG. 9E compares the processing time between using GPU (solid line) and CPU (dashed line). It shows that the CPU which is comparably more economical than GPU takes a shorter time to process when the size of signal batch is less than 10 which covers majority of the situation as shown in FIG. 9B.

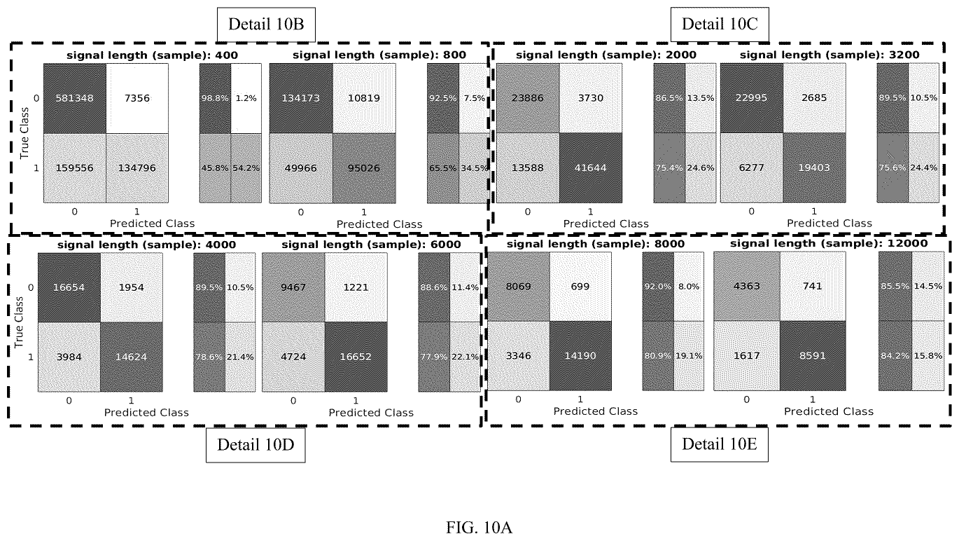

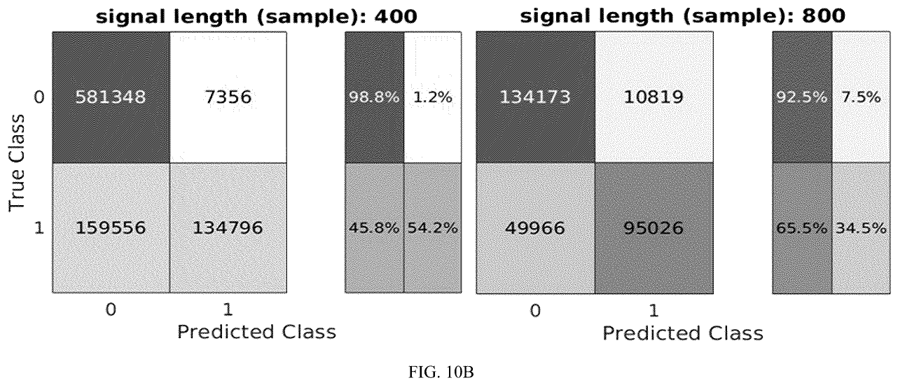

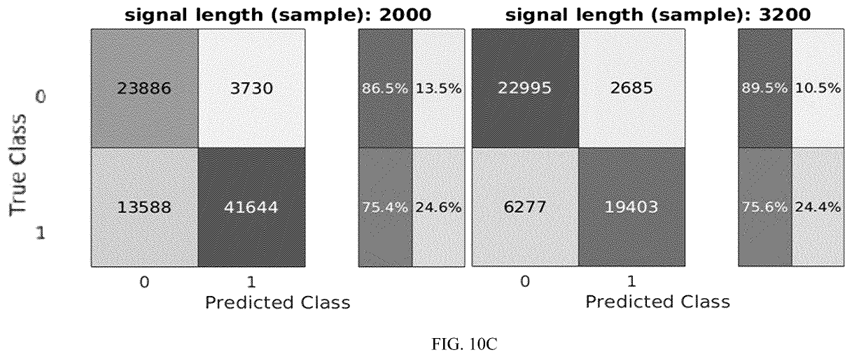

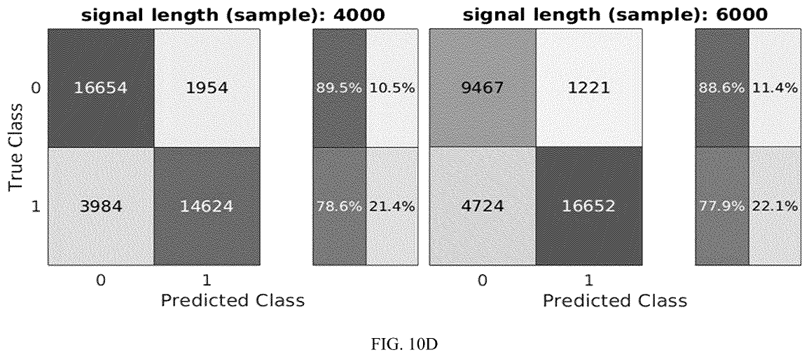

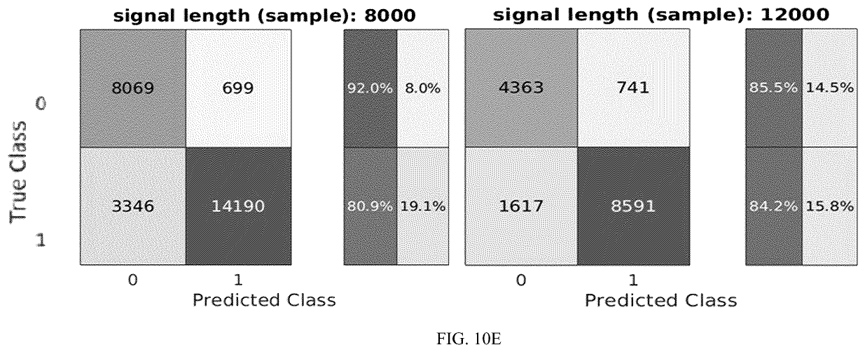

FIGS. 10A-10E illustrate confusion matrices of the LSTM neural network trained with various signal lengths for nanopore pathogen DNA selective sequencing in accordance with an embodiment of the subject invention. Label 0 means that the DNA read should be kept as it originated from pathogen of interest while label 1 means that it should be ejected. The confusion matrix is divided into four quadrants; the upper left of the matrix denotes the number of reads which should be kept and are actually kept by the LSTM neural network; the upper right denotes the number of reads which should be kept but are actually ejected by LSTM; the lower left denotes the number of reads which should be ejected but are actually kept by LSTM; the lower right denotes the number of reads which should be ejected and are actually ejected by LSTM. The color of each quadrant is normalized by the row. For the two quadrants on the upper left and lower right corners, the darker color indicates higher prediction performance, while the lighter colors indicate lower performance. The opposite is true for the other quadrants. To facilitate visualization of the result, a transformed confusion matrix is displayed to the right of the main confusion matrix. In this transformed matrix, the locations of the two lower quadrants are switched, and the numbers are normalized to percentages by the row. These graphs show that as the length of the signal increases, so does the prediction performance of the network where 4000 samples is equal to 1 second of the signal.

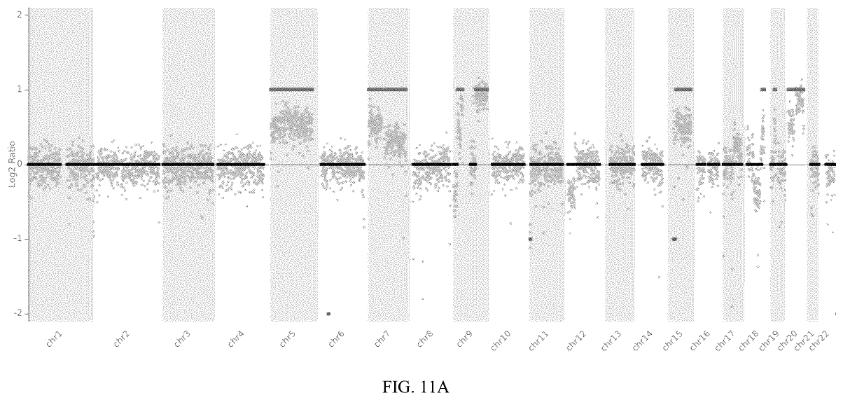

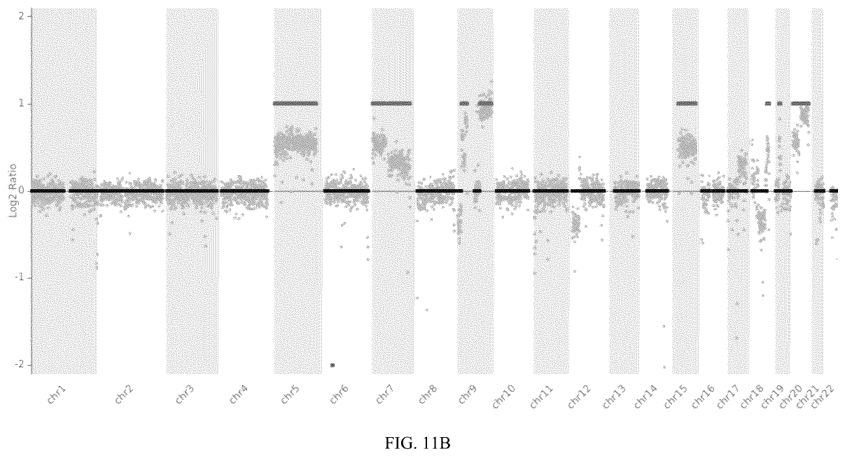

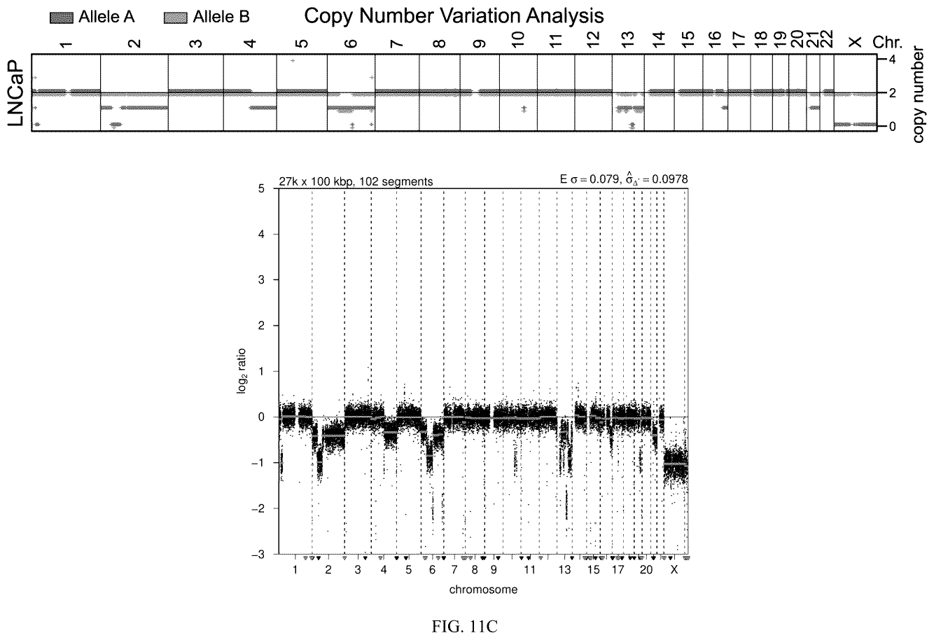

FIGS. 11A-11B illustrate Copy Number Variation (CNV) analysis differences between standard nanopore DNA sequencing without selective sequencing (FIG. 11A) and with selective sequencing (FIG. 11B) showing that it is feasible to conduct CNV analysis while conducting selective sequencing. FIG. 11A is generated using standard nanopore DNA sequencing data which lasts for 67.5 hours, capturing 3,044,811 DNA reads, the genome coverage of the genome is 4.07 times, the median length of reads is 5,981 bp. FIG. 11B is generated using selective sequencing data which lasts for 60 hours, targeting 18,100 non-overlapping genomic regions which size is about 10% of the genome. Since reads from non-target regions are ejected, 6,672,834 DNA reads were captured, the genome coverage is 2.06 times, the median length of the reads is 2,281 bp. Apart from a few small differences, the CNV analysis result in FIG. 11B is highly similar with the result in FIG. 11A and both results are correlated with the existing findings.

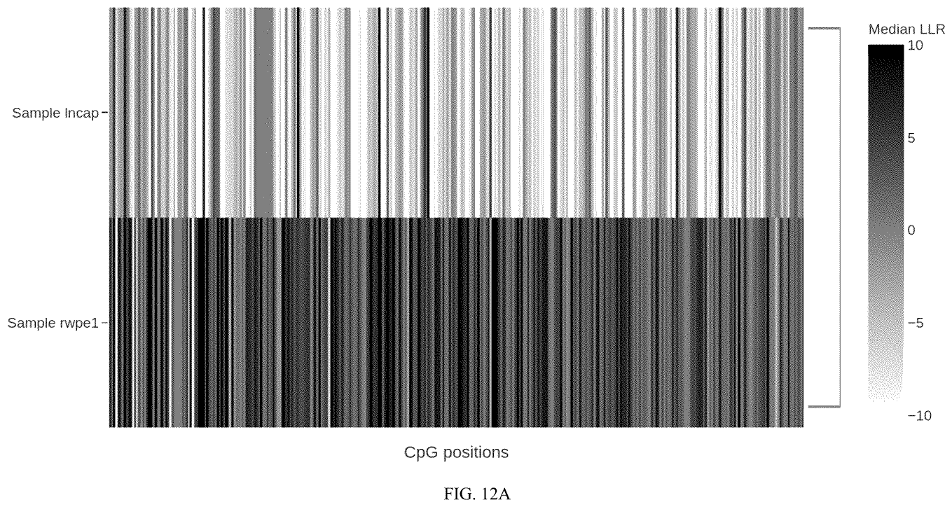

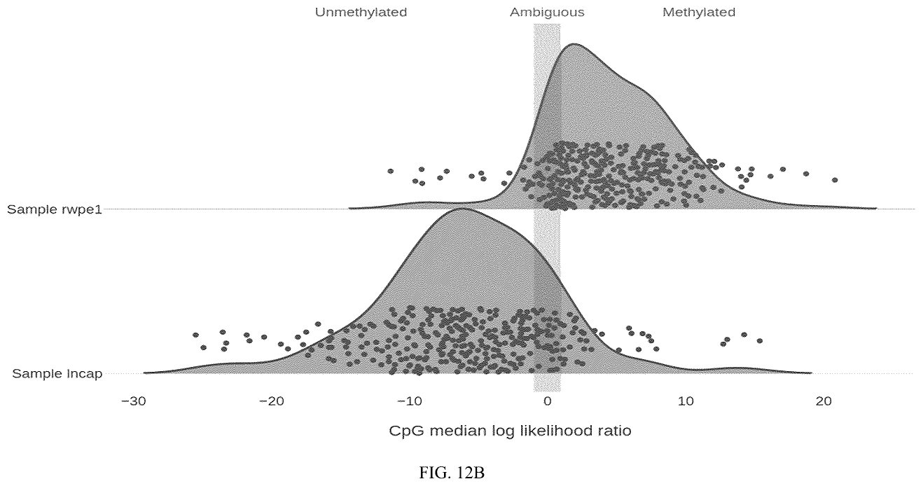

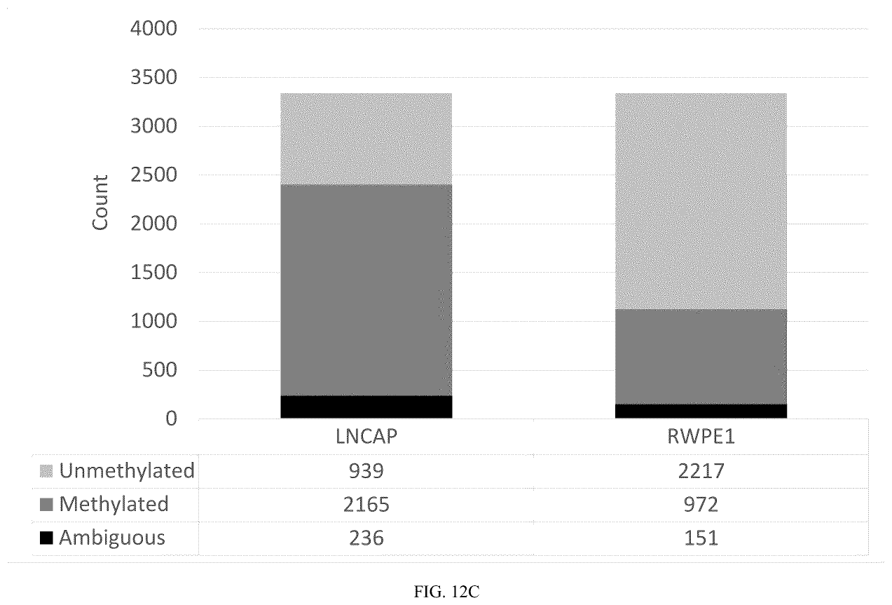

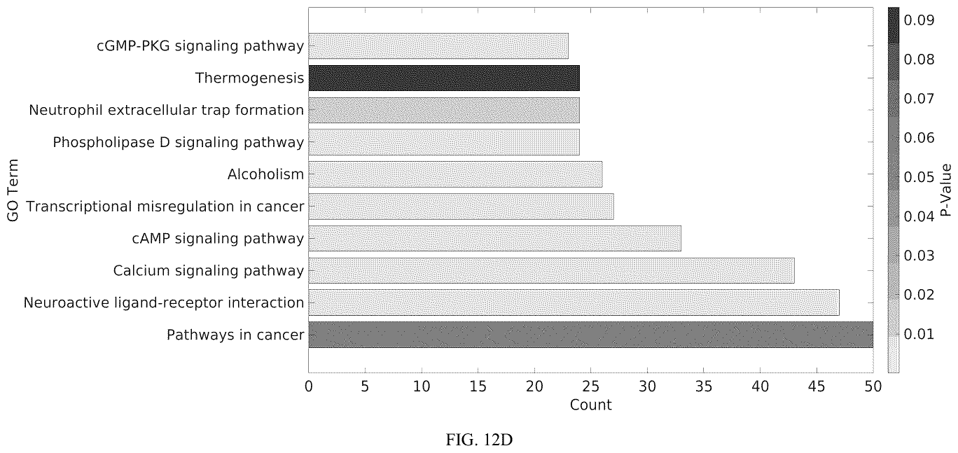

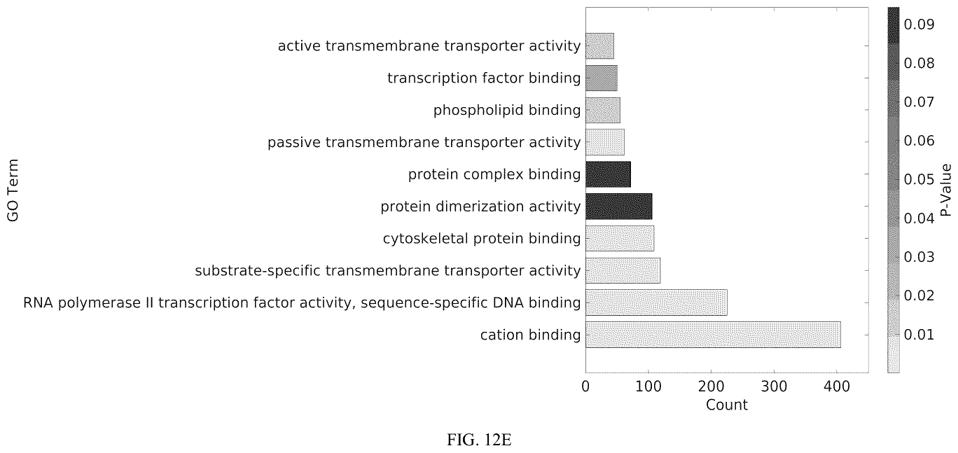

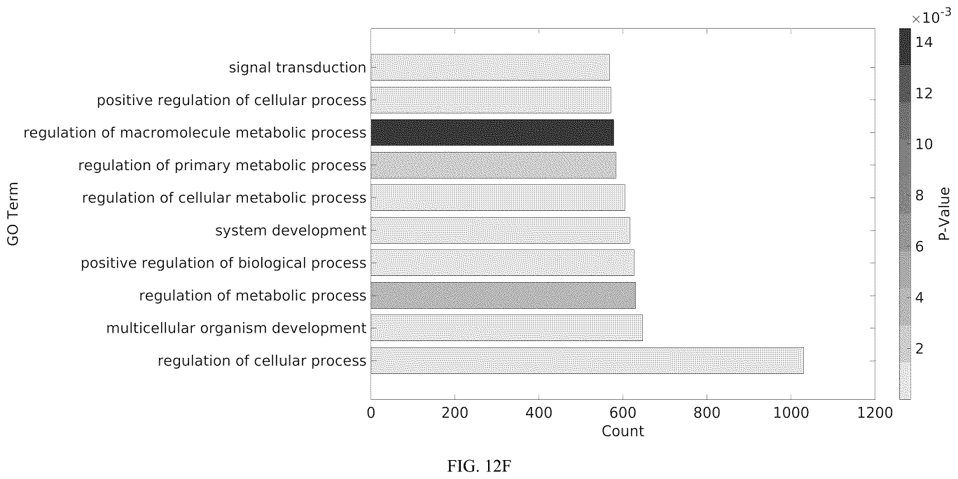

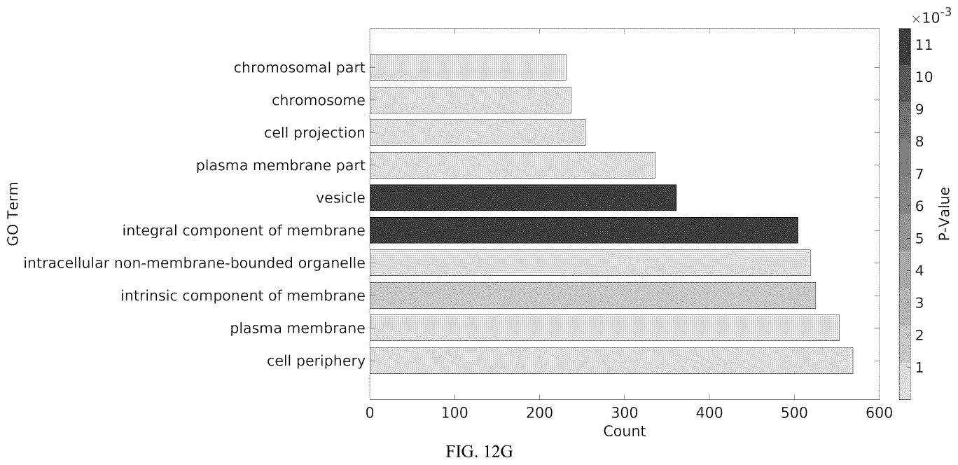

FIGS. 12A-12C illustrate DNA methylation analysis differences between LNCAP (prostate cancer cell line) and RWPE1 (normal prostate cell line) on a CpG island of ˜12.3 kb in length, containing 336 CpG sites. CpG sites are DNA sequences consisting of a cytosine nucleotide followed by a guanine nucleotide, connected by a phosphodiester bond. Methylation of CpG islands is often associated with gene silencing and can have important implications for disease processes, such as silencing of tumor-repressive genes in cancer. FIG. 12A illustrates the median LLR of all the 336 CpG sites in heatmap, the higher LLR the more likely this CpG site is methylated. The goal of nanopore DNA methylation analysis is to detect the presence of methylated cytosines and determine their location within the DNA sequence. To determine if CpG site on the DNA sequences mapped to this CpG island is methylated, the electrical signal around each site is analyzed by a statistical model because the signal of methylated cytosines is statistically different from unmethylated. Therefore, each CpG site is assigned with an LLR value denoting the probability of being methylated.

FIG. 12B illustrates the threshold for the determination of one of three potential status values of each site in ridge plot, if median LLR is less than −1, the site is classified as unmethylated; if median LLR is greater than 1, the site is classified as methylated; and if median LLR is between −1 and 1 (inclusive) the site is classified as ambiguous.

FIG. 12C illustrates the overall statistic of differently methylated CpG islands identified between LNCAP and RWPE1. The result is consistent with existing findings that methylation percentages in healthy tissues were much lower (Maruyama, Toyooka et al. 2002).

FIGS. 12D-12G illustrate the results of Gene Ontology (GO) analysis performed on differentially methylated CpG sites between LNCAP and RWPE1 cells. Specifically, FIG. 12D presents the KEGG pathway analysis results, showing that most of the differentially methylated CpG sites are associated with cancer pathways, followed by neuroactive ligand-receptor interactions. The involvement of neuroactive ligand-receptor interactions is significant, as these pathways can influence the behavior of prostate cancer cells by modulating hormonal responses and neural growth factors. FIG. 12E depicts the results for Molecular Function (MF), which is dominated by cation binding. Cation binding is crucial as it affects cellular processes such as signal transduction and enzyme activity, which are pivotal in the regulation and progression of prostate cancer. FIG. 12F depicts the Biological Process (BP) results, highlighting that “regulation of cellular process” accounts for a significant portion of the findings. This category is pivotal as it encompasses a range of mechanisms that control cell function and homeostasis, which are essential for maintaining cellular integrity and responding to oncogenic stress in prostate cancer development. FIG. 12G depicts the Cellular Component (CC) results, with “cell periphery” and “plasma membrane” emerging as the top findings. The “cell periphery” refers to the outermost region of the cell, critical for interactions with the cell's environment, while the “plasma membrane” is essential for cellular communication, nutrient uptake, and response to external signals, all of which are key in cancer cell dynamics.

### Exemplified Embodiments

The invention may be better understood by reference to certain illustrative examples, including but not limited to the following:

Embodiment 1. A system for providing a selective sequencing service over a communications network, the system comprising a frontend and a backend;

the frontend configured and adapted to:
a) collect a DNA/RNA read comprising a multiplicity of signals from one or more sequencer(s), the multiplicity of signals comprising a first electrical signal, a second electrical signal, and a third electrical signal,
b) trim each of the first electrical signal, the second electrical signal, and the third electrical signal, respectively, to form a trimmed signal set comprising a first trimmed signal, a second trimmed signal, and a third trimmed signal,
c) multiplex the trimmed signal set to form a multiplexed signal batch packet,
d) compress the multiplexed signal batch packet to form a compressed signal batch packet, and
e) transmit the compressed signal batch packet over the communications network to the backend; and
f) command the one or more sequencers to eject unwanted DNA/RNA according to a prediction result returned from the backend;

the backend configured and adapted to:
g) receive the compressed signal batch packet over the communications network from the frontend,
h) decompress the compressed signal batch packet to recover the multiplexed signal batch packet,
i) demultiplex the multiplexed signal batch packet to recover the trimmed signal set,
j) extract the trimmed signal set to recover the first trimmed signal, the second trimmed signal, and the third trimmed signal,
k) process each of the first trimmed signal, the second trimmed signal, and the third trimmed signal, respectively, to create an intermediate result set comprising a first intermediate result corresponding to the first electrical signal, a second intermediate result corresponding to the second electrical signal, and a third intermediate result corresponding to the third electrical signal,
l) analyze the intermediate result set to determine the prediction result of keep or eject for the DNA/RNA read, and
m) return the prediction result to the frontend over the communications network.

Embodiment 2. The system according to Embodiment 1, the one or more sequencers comprising one or more nanopore sequencers, each producing one or more respective nanopore sequencer electrical signals.

Embodiment 3. The system according to Embodiment 2, the backend comprising one or more neural networks trained for determining an origin of the DNA/RNA read from a nanopore sequencer electrical signal.

Embodiment 4. The system according to Embodiment 3, the communications network comprising at least one of a local network running on a single computer, a local network running between two or more computers, a private network running between two or more computers, or a virtual private network segment network running between two or more computers.

Embodiment 5. The system according to Embodiment 3, the communications network comprising a public internet segment.

Embodiment 6. The system according to Embodiment 4, the communications network comprising a public internet segment.

Embodiment 7. The system according to Embodiment 6, the one or more nanopore sequencers comprising a single nanopore sequencer configured and adapted to produce each of the first electrical signal, the second electrical signal, and the third electrical signal, respectively, in sequential order.

Embodiment 8. The system according to Embodiment 6, the one or more sequencers comprising a multiplicity of nanopore sequencers, each connected to a respective flowcell.

Embodiment 9. The system according to Embodiment 8, the multiplicity of nanopore sequencers, comprising a first nanopore sequencer, a second nanopore sequencer, and a third nanopore sequencer, respectively configured and adapted to produce each of the first electrical signal, the second electrical signal, and the third electrical signal, respectively, in parallel, and the frontend configured and adapted to multiplex a multiplicity of concurrent reads together in the multiplexed signal batch packet.

Embodiment 10. The system according to Embodiment 6, the DNA/RNA read comprising time series electrical current signal.

Embodiment 11. The system according to Embodiment 9, wherein the number of concurrent reads per flow cell is less than or equal to 10.

Embodiment 12. A neural network for determining an origin of a DNA/RNA read from a nanopore sequencer electrical signal, the neural network comprising:

an input layer;

a first LSTM layer;

a first dropout layer;

a second LSTM layer;

a second dropout layer;

a fully-connected layer;

a ReLu layer;

a Softmax layer; and

a classification layer.

Alternative to Embodiment 12. A 1D-CNN layer and Max Pooling 1D layer or a series of 1D-CNN layer and MaxPooling1D layer may be added in between the input layer and first LSTM layer to capture the extract relevant features from the input sequence. However, this alternative increases computational complexity, processing delays as well as hardware costs.

Embodiment 13. The neural network according to Embodiment 12, wherein the neural network is trained using collected nanopore sequencing signals.

Embodiment 14. The neural network according to Embodiment 13, wherein the first LSTM layer comprises 128 neurons and the second LSTM layer comprises 64 neurons.

Embodiment 15. The neural network according to Embodiment 14, wherein the first dropout layer, the second dropout layer, or both have a dropout probability set to about 0.25 to inhibit the neural network from overfitting.

Embodiment 16. The neural network according to Embodiment 15, wherein the neural network is configured and adapted to expand itself after processing a specified number of signals.

Embodiment 17. The neural network according to Embodiment 16, wherein the neural network is optimized with hyperparameter tuning based on a Bayesian Optimization method.

Embodiment 18. The neural network according to Embodiment 17, wherein the hyperparameter tuning is automated to increase the accuracy of the neural network.

Embodiment 19. The neural network according to Embodiment 13, wherein a majority of the collected nanopore sequencing signals used for training have a sample signal length between about 300 nucleotides and about 500 nucleotides.

Embodiment 20. A system for providing a selective sequencing service over a communications network, the system comprising a frontend and a backend;

the frontend configured and adapted to:
a) collect a DNA/RNA read comprising a multiplicity of signals from one or more sequencers, the multiplicity of signals comprising a first electrical signal, a second electrical signal, and a third electrical signal,
b) trim each of the first electrical signal, the second electrical signal, and the third electrical signal, respectively, to form a trimmed signal set comprising a first trimmed signal, a second trimmed signal, and a third trimmed signal,
c) multiplex the trimmed signal set to form a multiplexed signal batch packet,
d) compress the multiplexed signal batch packet to form a compressed signal batch packet, and
e) transmit the compressed signal batch packet over the communications network to the backend, and
f) command the one or more sequencers to eject unwanted DNA/RNA according to a prediction result returned from the backend;

the backend configured and adapted to:
g) receive the compressed signal batch packet over the communications network from the frontend,
h) decompress the compressed signal batch packet to recover the multiplexed signal batch packet,
i) demultiplex the multiplexed signal batch packet to recover the trimmed signal set,
j) extract the trimmed signal set to recover the first trimmed signal, the second trimmed signal, and the third trimmed signal,
k) process each of the first trimmed signal, the second trimmed signal, and the third trimmed signal, respectively, to create an intermediate result set comprising a first intermediate result corresponding to the first electrical signal, a second intermediate result corresponding to the second electrical signal, and a third intermediate result corresponding to the third electrical signal,
l) analyze the intermediate result set to determine the prediction result of accept or reject for the DNA/RNA read, and
m) return the prediction result to the frontend over the communications network;

the one or more sequencers comprising one or more nanopore sequencers, each producing one or more respective nanopore sequencer electrical signals;

the backend comprising one or more neural networks trained for determining an origin of the DNA/RNA read from a nanopore sequencer electrical signal;

the communications network comprising a private network or virtual private network segment, and a public internet segment;

the one or more nanopore sequencers comprising a first nanopore sequencer, a second nanopore sequencer, and a third nanopore sequencer, respectively configured and adapted to produce each of the first electrical signal, the second electrical signal, and the third electrical signal, respectively, in parallel, and the frontend configured and adapted to multiplex a number of concurrent reads together in the multiplexed signal batch packet;

the number of concurrent reads for one flow cell being less than or equal to 126 for a Flongle flowcell, less than or equal to 512 for a Minion flowcell, or less than or equal to 2675 for a PromethION or any other flowcell;

the DNA/RNA read comprising time series electrical current data;

the neural network comprising:
an input layer,
a first LSTM layer,
a first dropout layer,
a second LSTM layer,
a second dropout layer,
a fully-connected layer,
a ReLu layer,
a Softmax layer, and
a classification layer;

wherein the neural network is trained using collected nanopore sequencing signals;

wherein the first LSTM layer comprises 128 neurons and the second LSTM layer comprises 64 neurons;

wherein the first dropout layer, the second dropout layer, or both have a dropout probability set to about 0.25 to inhibit the neural network from overfitting;

wherein the neural network is configured and adapted to expand after processing a specified number of signals;

wherein the neural network is optimized with hyperparameter tuning using Bayesian optimization;

wherein the hyperparameter tuning is automated; and

wherein a majority of the collected nanopore sequencing signals used for training have a sample signal length between about 400 nucleotides and about 800 nucleotides.

### Materials and Methods

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1

### Realtime Selective Sequencing of Genomic Regions of Interests

There has been a growing expectation among cancer patients to receive personalized treatment based on the genomic signature of their tumor for better treatment outcomes. The most cost-effective solution would be sequencing only the genes or genomic regions of interests. However, related art NGS-based selective sequencing requires a sophisticated probe design and library preparation process. In addition, it also requires high sequencing depth for structural variation (SV) detection and a separate library to be generated for DNA methylation, both of which affects cancer progression, treatment outcomes, and other elements impacting treatment outcomes (Shridhar, Walia et al. 2016, Yamashita, Hosoda et al. 2018). In comparison, apart from selective sequencing of genomic regions of interests, embodiments allow the investigation of DNA methylation CNV and SV in one sequencing run without protocol modifications.

### Training Dataset Preparation

A neural network model needs to be trained for the selective sequencing of specific genomic regions with steps as follows. In certain embodiments it is contemplated that these steps are not to performed by end-users, but rather the end users can be provided an option to select pre-trained neural networks according to their requirements.

### Step 1

### FAST5 File Processing

FAST5 files recorded and now comprise the raw electrical signals of each respective DNA/RNA read moving across the pore. The signal contains information about the DNA/RNA sequence and its modifications, such as 5 mC or m6A. Each signal is assigned a unique read ID for identification purposes. To compile the training dataset for the neural network, the origin of each read needs to be determined first. In this example, a collection of FAST5 files is collected from the nanopore DNA sequencing on prostate cancer cell line LNCAP.

The first step of FAST5 processing is to convert FAST5 files to conventional FASTQ files through a process known as base calling (in this example, using a software called GUPPY.) The second step is to align the FASTQ files against reference genomes using software such as MINIMAP2 which is specialized for long reads alignment (Li 2018). The third step is alignment quality control, which can be the most important step. After the first two steps, sections of a DNA/RNA read can be mapped to different reference genomes or regions because of the similarity across the references or alternative splicing events. The number of matched bases of a read against the reference genome is used for calculating the map quality scores using an empirical formula which measures how well a read is mapped to the reference (Sherstinsky 2020). Because the formula contains a logarithmic function and a constant, the range of map quality score is [0,60]. To improve the robustness of the result in this example, only reads with map quality larger than 55 are selected. If one read has multiple mappings with same map quality score, such read is usually discarded. The final step is to extract the ID and alignment result of the reads which passed quality control (e.g., map quality larger than 55) for further processing.

### Step 2

### Raw Electrical Signal Extraction from FAST5

The first step of raw electrical signal extraction from FAST5 is to scan the structure of the FAST5 file using an h5info function from MATLAB R2022b or equivalent. The h5info function returns structural information about an entire FAST5 file, such as the read groups, datasets, and named datatypes contained within. By default, each FAST5 file contains 4,000 raw electrical signals, each signal is kept separately in a read group in FAST5. Apart from the electrical signal itself, each group also contains attributes such as the read ID, sampling rate, signal length, channel of recording, and additional attributes. The signal and attributes are extracted using “h5read” function.

The second step is sorting the extracted signal according to their read ID. This is necessary because the IDs are randomly generated during sequencing. Sorting can accelerate locating designated reads with read ID obtained from step 1.

The third step is saving the raw electrical signal of designated reads along with other information such as their origin.

### Step 3

### Signal Slicing and Training Dataset Preparation

To compile the training dataset, the first step is trimming both the beginning and the end of the extracted signal. This trimming is beneficial because the beginning of the signal is expected to contain ˜30 nts of adapter sequences, and additional ˜20 nts of barcode sequences, if barcodes are added during library preparation as well as other auxiliary sequences such as poly(A). The last few nucleotides at the end of DNA/RNA can be sequenced faster than the rest due to the release of DNA/RNA from motor protein should also be removed (Workman, Tang et al. 2019).

The second step is signal slicing, performed by slicing electrical signals using a sliding window of various lengths. By default, the step of sliding window is half of the window length. This step improves the robustness of the dataset and helps to find the balancing point between input signal length and neural network performance.

In the end, sliced signals are grouped according to their length, labelled accordingly and reshuffled to finish the training dataset compiling, 20% of the dataset is reserved for validation. Instead of saving the sliced signals and their labels in separate files, it can be more efficient to store multiple sliced signals and labels in one csv or tsv file as it can increase disk input/output performance compared to storing in separate files. In this example, the regions of interest are the CpG islands on the human genome plus 3,000 nt of flanking regions. In certain embodiments, the length of the flanking regions is adjusted according to factors such as the average length of the DNA/RNA reads and clinical requirements.

### Neural Network Architecture

The key components of the neural network are two LSTM layers wherein first LSTM layer has 128 neurons while the latter has 64 neurons. Long Short Term Memory Networks (LSTMs) have the advantage of being able to remember information over long-term contexts. Unlike traditional neural networks, they are capable of capturing long-term dependencies in data and using them accurately when making predictions. This is because LSTMs contain self-loops that read and store information from prior states. This form of memory allows LSTMs to capture patterns in sequences of data over multiple time steps, increasing the accuracy and complexity of their predictions as compared to vanilla recurrent neural networks. Each LSTM neuron is influenced by all the neurons before it and therefore can capture long-term dependencies among the signal; the accompanying dropout layer (e.g., with dropout probability set to 0.25) can inhibit the neural network from overfitting (Yu, Si et al. 2019, Sherstinsky 2020). In the end of the neural network are a Softmax layer and classification layer which outputs the prediction result. The provided network architecture is different from the 1D-ResNet used by SquiggleNet (Bao, Wadden et al. 2021). Instead of having a fixed network architecture, LSTM neural network can expand itself as more signals are fed into the network.

### Hyperparameter Tuning Using Bayesian Optimization

Apart from proper network architectures, it is often also a challenge to optimize all the key hyperparameters such as loss function, weight initialization method, and learning rate to obtain satisfying network performance. Fortunately, with the help of hyperparameters tuning methods such as Bayesian Optimization (BO) and Automated Machine Learning (Auto-ML), hyperparameter tuning can be automated (Sherstinsky 2020). BO is often used for finding the minimum or maximum of a function which is expensive to evaluate by iteratively searching the most promising points based on a probabilistic model of the function. It is particularly useful in machine learning and optimization problems where the objective function is complex and expensive to evaluate, such as hyperparameter tuning, neural architecture search. BO employs a probabilistic model of Gaussian process, which creates a surrogate function that predicts the values of the objective function at untested points. BO then uses an acquisition function to determine the next point to evaluate, balancing between exploitation of the point of best performances and exploration of untested regions of the search space. The process searches iteratively until convergence, resulting in an estimate of the optimal point of the function.

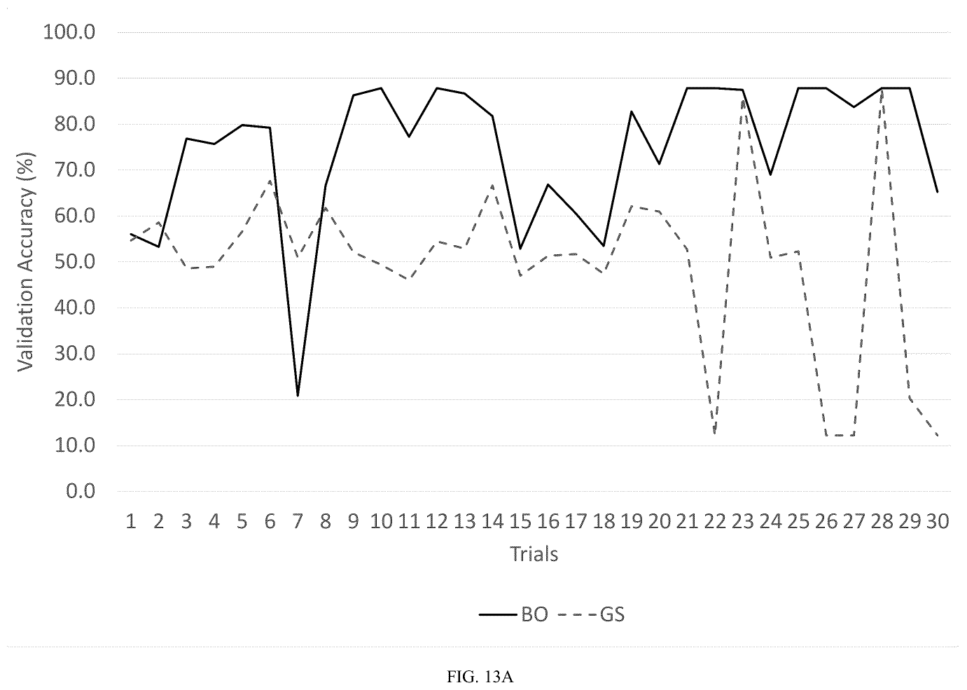

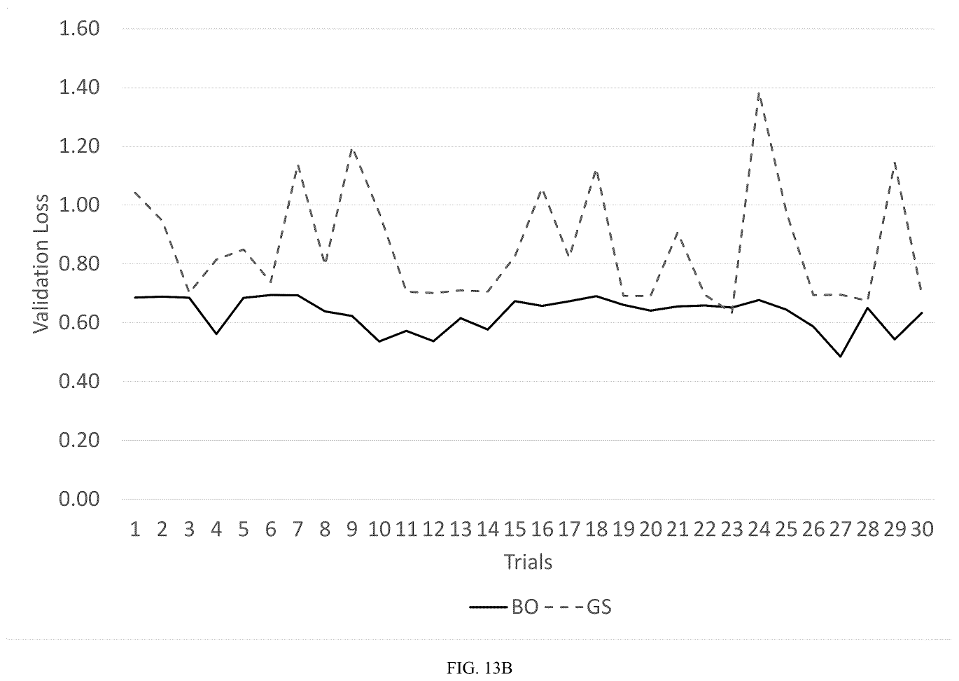

Grid Search (GS) and Randomized Grid-Search (RGS) are two common methods for hyperparameter optimization. In short, GS systematically scans through the hyperparameters and finds the combination which results in best performance while RGS tries hyperparameters randomly. In comparison, neural network's performance is modeled as a sample from a gaussian process in BO. By tuning hyperparameters based on the network's performance from last iteration accordingly, BO provides greater automation and finds better hyperparameters significantly faster than other approaches (Snoek, Larochelle et al. 2012, Shahriari, Swersky et al. 2015). GS and BO are employed to conduct tunning of four hyperparameters: (i) learning rate, (ii) Number of neurons in “LSTM layer 1” illustrated in FIG. 2A, (iii) Number of neurons in “LSTM layer 2” illustrated in FIG. 2A and (iv) input signal normalization method. The learning rate is a crucial hyperparameter that affects how quickly the model learns and converges to the optimal solution, when learning rate is low, the neural network may take longer to train, and it may get stuck in a suboptimal solution; on other hand, if the learning rate is too high, the neural network may overshoot and fail to train. Likewise, the number of neurons affects the generalization ability of the neural network, more neurons take longer to train while fewer neurons may fail to achieve desirable performance. The “validation accuracy” is a metric indicates how well the neural network can generalize to unseen data in the validation dataset, the higher the validation accuracy, the better. The “validation loss” is another performance metric which is opposite to validation accuracy, should be minimized. As illustrated in FIG. 13A-FIG. 13B, BO achieves validation accuracy of 87.82% (solid line) and validation loss of 0.5364 after 10 trials and then gradually converges to similar performances around 87% in the subsequent trails. In comparison, GS (dashed line) takes 28 trails to reach a similar validation accuracy of 87.68% but higher validation loss of 0.6743. Proving the effectiveness of BO in hyperparameter tunning. In certain embodiments, additional hyperparameters such as “activation function” and “dropout probability of the dropout layers” even “number of LSTM layers” can also be added into Bayesian optimization. While conventional optimization goal is to maximize validation accuracy of the network, it is also possible to include “processing efficiency” in the optimization goal.

### Library Preparation & Experiment Setup

The libraries of different concentration are prepared using DNA extracted from LNCAP prostate cancer cell line following the recommendation procedure of standard nanopore DNA library preparation kit LSK-SQK110. The library is sequenced with MINION flowcells (R9.4.1) using nanopore sequencer MIN-101B. The experiment setup is identical to the configuration in FIGS. 1A-1B, ejecting reads which do not originate from genomic region of interest. To ensure the timely processing of the electrical signals, the backend is initialized, and the neural network is loaded into the computer memory before the sequencing run begins.

### Selective Sequencing Performance Evaluation

As illustrated in FIGS. 7A-7C, the selective sequencing successfully achieves higher enrichment in target region than non-target region. High library concentration improves the enrichment performance of selective sequencing. In summary, selective sequencing can increase the efficiency of nanopore sequencing and thus lowering the sequencing cost.

As illustrated by FIG. 7A, the increment in coverage of target region (solid line) and non-target region (dashed line) is high during first 15 hours and gradually declines as the percentage of active pores in the flowcell (dotted line) decreases. Because the lifespan of the flowcell used by nanopore sequencing is limited, the flowcell can wear out during one sequencing run and hence reduce the yield. While it is possible to revitalize the flowcell to reduce the sequencing cost, revitalization can only be performed after sequencing. Therefore, during one sequencing run, both the percentage of active pores and the increment in coverage of both target and non-target region, can each, respectively, decline.

FIGS. 7B-7C illustrate the influence of library concentration on enrichment performance of selective sequencing according to an embodiment of the subject invention. As illustrated by FIG. 7B, the enrichment of target region over the non-target region can also decrease and then stabilize rather than continue increasing during the sequencing run. This is because the working principle of selective sequencing is ejected DNA/RNA reads from uninteresting genomic regions or species not actively capturing the reads of interests. As illustrated by FIG. 7C, increasing the library concentration results in higher enrichment over non-target regions and a greater percentage of active pores. Specifically, when library concentration is 40 fmol, the enrichment is about 3 (solid line), it is about 1.43 times higher when library concentration is 10 fmol (solid line with diamond marker). Furthermore, the percentage of active pores increases with higher library concentration, when library is 40 fmol, about 62% of the pores are active at the beginning of the sequencing run and 15% of the pores remain active after 35 hours of sequencing (dashed line). In contrast, when the library concentration is 10 fmol, there are only about 28% of the pores are active at the beginning of the sequencing and only 3% of the pores that are still active after 35 hours (dashed line with circle marker). The figure also suggests that higher library concentrations prolong the turning point for the enrichment performance. For example, the turning point is extended for about 10 hours for higher library concentration (e.g., 40 fmol) compared to lower library concentration (e.g., 10 fmol).

This demonstrates that the library concentration is one of the factors which influence the enrichment performance of selective sequencing. Higher library concentration is beneficial towards selective sequencing, as it increases the percentage of active pores and the enrichment performance and thus shortens the turnaround time.

Copy Number Variation (CNV) analysis after selective sequencing

Copy Number Variation (CNV) refers to the variation in the number of copies of a specific segment of the genome. CNV such as deletions and duplications alters transcription of genes by altering the dosage or disrupting proximal or distant regulatory regions (Shlien and Malkin 2009). Thus, CNV is often related to genetic diseases and cancer as well. For example, CNV around the gene FMR1 (fragile X mental retardation 1) gene is often related with fragile X syndrome, a developmental disorder that affects cognitive and behavioral functions (Nagamani, Erez et al. 2012). In prostate cancer, CNV is associated with increased risk of metastases at diagnosis (Grist, Friedrich et al. 2022).

To conduct CNV analysis, the FAST5 files are processed using the FAST5 file processing stipulated in EXAMPLE 1. Next, the genome is partitioned into nonoverlapping, fixed-sized bins of 100 kb. For each bin, various statistics such as the GC content in the reference genome, the number of reads mapped within the bin, the mapping quality of the reads were collected and adjusted. These statistics are employed to determine the occurrence of deletions or duplications which cause fewer or more reads in a bin, respectively.

As illustrated in FIGS. 11A-11B, whole genome CNV analysis is also conducted after selective sequencing where DNA reads from non-target genomic region are ejected. These ejected reads are partially sequenced and recorded before being ejected. The recorded length of ejected reads (500˜600 bp) is longer than related art systems and methods such as NGS (150 bp). Therefore, with only genome coverage of two times, it is possible for selective sequencing to achieve highly similar result as the standard nanopore sequencing which genome coverage is four times.

### Differentially Methylated Region (DMR) Analysis After Selective Sequencing

As illustrated in FIGS. 12A-12C, whole genome DMR analysis is conducted after selective sequencing. In addition to the nanopore selective sequencing data on LNCAP in this example, separate nanopore sequencing data on RWPE1 cell line (normal prostate) is collected following the same procedure but without any selective sequencing as reference for DMR analysis. A CpG island located in human chromosome chr15: 101752508-101764820, containing 336 CpG sites is selected as example.

The first step of DMR analysis is obtaining the median LLR (Log-Likelihood Ratio) of each CpG site in the CpG island. LLR is a statistical measure used to calculate the probability of a given CpG site being methylated or unmethylated, the higher the LLR value, the more likely the CpG site is to be methylated.; Compared to mean value, median value is less susceptible to the influence of extreme values. As illustrated in FIG. 12A, the median LLR values of the CpG sites in the example CpG island are plotted in heat plot. The x-axis represents the position of the CpG site in the CpG island, while the y-axis represents the median LLR value.

The second step is to determine if each site CpG is methylated or unmethylated or ambiguous by using a threshold. FIG. 12B illustrates the distribution of median LLR value of the CpG sites in the CpG island in violin plot. For CpG sites whose mean LLR falls within the threshold are regarded as “ambiguous” (indicated by the light grey boundary box) while those above and below the threshold are classified as “methylated” and “unmethylated” respectively.

Next, Mann-Whitney U test is employed to test if this CpG island there is a DMR. Mann-Whitney U test is subject to less stringent assumptions than the t-test and thus selected because the requirement of normal distribution for the t-test is not met in this example. Finally, the p-value is adjusted using Benjamin & Hochberg (BH) method. The BH adjusted p-value of the Mann-Whitney U test for this CpG island is 4.862687e-76 which is below the threshold and thus the null hypothesis: this CpG island is not a DMR is rejected and considered as a DMR between RWPE1 and LNCAP.

In the end, methylation status counts for all identified DMR in the human genome visualized in stacked column plots (FIG. 12C). It illustrates that the CpG islands in the prostate cancer cell line (LNCAP) are more methylated than the prostate normal cell line (RWPE1) which is consistent with existing findings. The GO analysis results are shown in FIGS. 12D-12G.

### Example 2

### Realtime Pathogen Enrichment in Clinical Samples

Existing pathogen detection methods, such as tissue culturing and PCR-based kits, have certain limitations. Tissue culturing is a labor-intensive process which can require several days to complete, which makes it unsuitable for use in emergency rooms or other time-sensitive settings. In contrast, current embodiments of the subject invention have been shown to reduce the turnaround time to about 2 hours. PCR-based pathogen detection methods can only detect limited types of pathogens. In comparison, embodiments that can capture long reads (e.g., greater than or equal to about 20 kb) had drastically reduced the required sequencing depth for pathogen identification through assembling pathogen genome as compared to related art, illumina-based approaches.

To demonstrate the performance of an embodiment of the subject invention, a real-time selective sequencing using the nasal swap samples from COVID-19 positive patients is conducted.

The training dataset preparation, neural network design and training is similar to the procedures stipulated in EXAMPLE 1 and therefore are omitted here.

### Library Preparation & Experiment Setup

To preserve RNA modification and shorten library preparation time, the library is prepared following the ONT's standard direct RNA sequencing protocol (i.e., LSK-RNA002) without adding the control RNA. The minion flowcell is divided into one control group and one experiment group where the experiment group enabled the selective sequencing and yeast originated reads are selectively ejected. Because it is known that the yeast is the dominant species in this kind of sample (˜97%).

### Detection and Removal of Poly(A) Tails

The presence of poly(A) tails is the most characteristic difference between nanopore direct RNA sequencing protocol and nanopore DNA sequencing. Poly(A) tails are formed during messenger RNA (mRNA) maturation which occurs during DNA transcription. After transcription, a poly(A) tail is added to the 3′ end of the RNA molecule to increase the stability of the molecule. As shown in FIG. 3A, the adapter sequence of standard direct RNA sequencing protocol contains a poly(A) tail. This allows RNA molecules with a poly(A) tail can ligate with the adapter during the library preparation and be sequenced. As a result, the electrical signal of the sequenced RNA always contains a section of poly(A) tail whose length varies due to various factors. However, due to its excessive length, poly(A) tail can hinder selective sequencing and should be trimmed before sending the signal for classification.

The trimming of poly(A) tail can be achieved by leveraging its two features: (i) the abrupt transition in electrical signal between the poly(A) and adapter; and (ii) the relative low standard deviation of the poly(A) signal. The PELT method is employed to segment the electrical signal (Killick, Fearnhead et al. 2012). The algorithm finds N change points (e.g., N can be a user specified number, a fixed system parameter, or a dynamic parameter) where the signal had changed drastically. Naturally, these change points would separate the signal into N+1 regions. As illustrated in FIGS. 4A-4D, when N=1, the algorithm would select the change point (vertical dashed line) which divides the signal into two regions that the residual squared error between the signal (solid line) in the corresponding region and the local mean (horizontal dashed line) of the region is minimized. If N>1, the steps above are repeated until the requirement on change points are met. When N=2, an advantageous segmentation performance of separating poly(A) and adapter is achieved.

### Selective Sequencing Performance Evaluation

As illustrated in FIG. 5, with the input signal length of 4 s (equivalent to ˜280 nt), the validation accuracy of LSTM neural network reaches 87.82% after training and BO hyperparameter tuning. Other methods such as logistic regression, decision tree, and KNN reach validation accuracies of 80.60%, 78.61% and 73.33% respectively.

As illustrated in FIGS. 6A-6E, with signal input length set to 4 seconds (equivalent to ˜280 nts), 1.73 times˜2.99 times of enrichment in coverage among various pathogens were observed in the experimental group. For example, 2.99 times of enrichment in staphylococcus aureus, a gram-positive spherically shaped bacteria which causes a wide variety of clinical diseases was observed. 2.88 times of enrichment in lactobacillus fermentum was observed. 2.01 times of enrichment in candida albicans, a naturally occurring fungus found on the human body but can cause infection when overproduced was observed. 1.73 times of enrichment in scarcer pathogens such as COVID-19 was observed. While not being bound by theory, it is hypothesized that there is a concentration limit of pathogen originated RNA to observe plausible enrichment fold in real-time pathogen enrichment in clinical samples as stated in EXAMPLE 1.

### Example 3

### Distinguishing Bacteria Originated DNA and Human Originated DNA

As illustrated in FIGS. 10A-10E, embodiments comprising a neural network for distinguishing bacteria originated DNA (label: 0) and human originated DNA (label: 1) in supplementary to EXAMPLE 2 are also tested. Data was downloaded from public sources. The training dataset is prepared using the same procedure above but sliced into various signal lengths (e.g., 4000 samples per second) to observe the influence of signal length on network performance.

### Example 4

### Depletion of Unwanted DNA/RNA

Another potential application of the invention is the depletion of unwanted DNA/RNA from specific genomic regions which are deemed unnecessary. This is particularly useful when the genomic regions of interest cannot be clearly defined. For example, ribosomal RNA (rRNA) constitutes approximately 90% RNA species in total RNA, it is detrimental to the whole transcriptome analysis and thus must be depleted (O'Neil, Glowatz et al. 2013). Despite the effort of polyA⁺ enrichment employed in the nanopore DRS protocol to remove rRNA, mitochondrial RNAs (mt-RNAs) and rRNA from RPLx family and RPSx family can still takes up ˜30% of the sequenced read after nanopore RNA sequencing, reducing the effective yield of the flowcell (Mercer, Neph et al. 2011, Mock, Braun et al. 2023). It is because certain mt-RNAs such as mitochondrial mRNA (mt-mRNA) and rRNA (mt-rRNA) are polyadenylated mt-rRNAs and thus cannot be removed during the polyA⁺ enrichment process (Slomovic, Laufer et al. 2005). Therefore, by ejecting mt-RNAs and rRNA during sequencing, the yield of RNA from other regions can be increased using selective sequencing.

### Example 5

### Nanopore Selective Sequencing as a Service

As illustrated in FIG. 8, real-time selective nanopore sequencing is divided into 5 stages. After DNA/RNA read enters the pore (T1˜T2), the adapter sequence is sequenced first and then the DNA/RNA is sequenced continuously (T2˜T3) even after the electrical signal is extracted for classification. Therefore, prolonged processing time (T4˜T5) is detrimental to the yield of selective sequencing and must be reduced. Employing powerful but expensive computer hardware for base calling and read alignment is not an economical solution for wide adoption of selective sequencing. The feasible solution is to provide nanopore selective sequencing in the form of Software as a Service (SaaS)

### Internet Infrastructure Analysis

For the embodiments to be provided as SaaS over the network, especially the Internet. Apart from lowering processing time, it is vital to reduce the transmission bandwidth requirement. As of May 2022, the global mean upload bandwidth of mobile Internet is 14.10 Mbps (74.96 Mbps for the wired Internet) and mean latency is 18 ms. As illustrated in FIGS. 9C-9D, when using there is ample bandwidth to provide selective sequencing over the Internet. Allowing users to conduct selective sequencing anywhere and anytime with no prior capital investment.

### Cost Efficiency, Reach, and Usability Analysis

The current total cost at time of filing for standard nanopore sequencing for one sample is about USD$600, including $450 for one flowcell and $100 for the reagents. (The cost of the sequencer is ˜USD$400 but often purchased as part of a start-up deal which includes a flowcell, a sequencer and some reagents). According to previous examples, embodiments of the subject invention can drastically increase the efficiency of one flowcell. Furthermore, the lifespan of a flowcell is also extended so that cost per sample can also be reduced by a factor of two or three in certain situations.

More importantly, embodiments can be implemented on CPUs (about 7.4 times cheaper than GPUs at the time of filing) for providing selective sequencing services. This can be translated into huge hardware cost savings for companies hosting such services.

As illustrated in FIGS. 9A-9E, according to the simulation test using the original data from the previous nanopore direct RNA sequencing, it is demonstrated that by processing reads in parallel, the sequencing throughput can increase up to 3.71 times. By multiplexing signals into one signal batch in frontend (e.g., about 5 signals per signal batch) and transmitting to the backend can save signal transmission bandwidth up to 2.98 times. In addition, the neural network design of certain embodiments gives host CPU (e.g., CPU model: AMD 5900X, single thread) advantages over GPU (e.g., GPU model: NIVIDIA 3090Ti) when the number of concurrent reads or signal batch size is lower than 10. This lowers the hardware requirement and the upfront cost up to about 7.4 times (e.g., at the time of filing, AMD 5900X can be purchased for approximately USD$340 while NVIDIA 3090Ti can be purchased for approximately USD$2500.)

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

ADDIN EN.REFLIST Bao, Y., J. Wadden, J. R. Erb-Downward, P. Ranjan, W. Zhou, T. L. McDonald, R. E. Mills, A. P. Boyle, R. P. Dickson and D. Blaauw (2021). “SquiggleNet: real-time, direct classification of nanopore signals.” Genome biology 22 (1): 1-16.
Branco, M. R., G. Ficz and W. Reik (2012). “Uncovering the role of 5-hydroxymethylcytosine in the epigenome.” Nature Reviews Genetics 13 (1): 7-13.
Cheng, Y., C. He, M. Wang, X. Ma, F. Mo, S. Yang, J. Han and X. Wei (2019). “Targeting epigenetic regulators for cancer therapy: mechanisms and advances in clinical trials.” Signal transduction and targeted therapy 4 (1): 62.
Daniunaite, K., S. Jarmalaite, N. Kalinauskaite, D. Petroska, A. Laurinavicius, J. R. Lazutka and F. Jankevicius (2014). “Prognostic value of RASSFI promoter methylation in prostate cancer.” The Journal of urology 192 (6): 1849-1855.
Ekblom, R. and J. Galindo (2011). “Applications of next generation sequencing in molecular ccology of non-model organisms.” Heredity 107 (1): 1-15.
Friedman, A. A., A. Letai, D. E. Fisher and K. T. Flaherty (2015). “Precision medicine for cancer with next-generation functional diagnostics.” Nature Reviews Cancer 15 (12): 747-756.
Gaudin, M. and C. Desnues (2018). “Hybrid capture-based next generation sequencing and its application to human infectious diseases.” Frontiers in microbiology 9:2924.
Grist, E., S. Friedrich, C. Brawley, L. Mendes, M. Parry, A. Ali, A. Haran, A. Hoyle, C. Gilson, S. Lall, L. Zakka, C. Bautista, A. Landless, K. Nowakowska, A. Wingate, D. Wetterskog, A. M. M. Hasan, N. B. Akato, M. Richmond, S. Ishaq, N. Matthews, A. A. Hamid, C. J. Sweeney, M. R. Sydes, D. M. Berney, S. Lise, M. K. B. Parmar, N. W. Clarke, N. D. James, P. Cremaschi, L. C. Brown and G. Attard (2022). “Accumulation of copy number alterations and clinical progression across advanced prostate cancer.” Genome Med 14 (1): 102.
Jain, M., S. Koren, K. H. Miga, J. Quick, A. C. Rand, T. A. Sasani, J. R. Tyson, A. D. Beggs, A. T. Dilthey and I. T. Fiddes (2018). “Nanopore sequencing and assembly of a human genome with ultra-long reads.” Nature biotechnology 36 (4): 338-345.
Jiang, Z., H. Wang, J. J. Michal, X. Zhou, B. Liu, L. C. S. Woods and R. A. Fuchs (2016). “Genome wide sampling sequencing for SNP genotyping: methods, challenges and future development.” International Journal of Biological Sciences 12 (1): 100.
Khamlichi, A. A. and R. Feil (2018). “Parallels between mammalian mechanisms of monoallelic gene expression.” Trends in Genetics 34 (12): 954-971.
Killick, R., P. Fearnhead and I. A. Eckley (2012). “Optimal detection of changepoints with a linear computational cost.” Journal of the American Statistical Association 107 (500): 1590-1598. Kit, A. H., H. M. Nielsen and J. Tost (2012). “DNA methylation based biomarkers: practical considerations and applications.” Biochimie 94 (11): 2314-2337.
Kovaka, S., Y. Fan, B. Ni, W. Timp and M. C. Schatz (2021). “Targeted nanopore sequencing by real-time mapping of raw electrical signal with UNCALLED.” Nature biotechnology 39 (4): 431-441.
Lagu, T., M. B. Rothberg, M.-S. Shich, P. S. Pekow, J. S. Steingrub and P. K. Lindenauer (2012). “Hospitalizations, costs, and outcomes of severe sepsis in the United States 2003 to 2007.” Critical Care Medicine 40 (3): 754-761.
Li, H. (2018). “Minimap2: pairwise alignment for nucleotide sequences.” Bioinformatics 34 (18): 3094-3100.
Mamanova, L., A. J. Coffey, C. E. Scott, I. Kozarewa, E. H. Turner, A. Kumar, E. Howard, J. Shendure and D. J. Turner (2010). “Target-enrichment strategies for next-generation sequencing.” Nature methods 7 (2): 111-118.
Maruyama, R., S. Toyooka, K. O. Toyooka, A. K. Virmani, S. Zöchbauer-Müller, A. J. Farinas, J. D. Minna, J. McConnell, E. P. Frenkel and A. F. Gazdar (2002). “Aberrant promoter methylation profile of prostate cancers and its relationship to clinicopathological features.” Clinical cancer research 8 (2): 514-519.
Mercer, T. R., S. Neph, M. E. Dinger, J. Crawford, M. A. Smith, A.-M. J. Shearwood, E. Haugen, C. P. Bracken, O. Rackham and J. A. Stamatoyannopoulos (2011). “The human mitochondrial transcriptome.” Cell 146 (4): 645-658.
Mock, A., M. Braun, C. Scholl, S. Fröhling and C. Erkut (2023). “Transcriptome profiling for precision cancer medicine using shallow nanopore cDNA sequencing.” Scientific Reports 13 (1): 2378.
Nagamani, S. C., A. Ercz, F. J. Probst, P. Bader, P. Evans, L. A. Baker, P. Fang, T. Bertin, P. Hixson and P. Stankiewicz (2012). “Small genomic rearrangements involving FMRI support the importance of its gene dosage for normal neurocognitive function.” Neurogenetics 13:333-339.
Nielsen, R., J. S. Paul, A. Albrechtsen and Y. S. Song (2011). “Genotype and SNP calling from next-generation sequencing data.” Nature Reviews Genetics 12 (6): 443-451.
O'Neil, D., H. Glowatz and M. Schlumpberger (2013). “Ribosomal RNA depletion for efficient use of RNA-seq capacity.” Current protocols in molecular biology 103 (1): 4.19. 11-14.19. 18.
Onay, V. U., L. Briollais, J. A. Knight, E. Shi, Y. Wang, S. Wells, H. Li, I. Rajendram, I. L. Andrulis and H. Ozcelik (2006). “SNP-SNP interactions in breast cancer susceptibility.” BMC cancer 6 (1): 1-16.
Payne, A., N. Holmes, T. Clarke, R. Munro, B. J. Debebe and M. Loose (2021). “Readfish enables targeted nanopore sequencing of gigabase-sized genomes.” Nature biotechnology 39 (4): 442-450.
Quail, M. A., M. Smith, P. Coupland, T. D. Otto, S. R. Harris, T. R. Connor, A. Bertoni, H. P. Swerdlow and Y. Gu (2012). “A tale of three next generation sequencing platforms: comparison of Ion Torrent, Pacific Biosciences and Illumina MiSeq sequencers.” BMC Genomics 13 (1): 341.
Rand, A. C., M. Jain, J. M. Eizenga, A. Musselman-Brown, H. E. Olsen, M. Akeson and B. Paten (2017). “Mapping DNA methylation with high-throughput nanopore sequencing.” Nature methods 14 (4): 411-413.
Roach, N. P., N. Sadowski, A. F. Alessi, W. Timp, J. Taylor and J. K. Kim (2020). “The full-length transcriptome of C. elegans using direct RNA sequencing.” Genome research 30 (2): 299-312.
Shahriari, B., K. Swersky, Z. Wang, R. P. Adams and N. De Freitas (2015). “Taking the human out of the loop: A review of Bayesian optimization.” Proceedings of the IEEE 104 (1): 148-175.
Sherstinsky, A. (2020). “Fundamentals of recurrent neural network (RNN) and long short-term memory (LSTM) network.” Physica D: Nonlinear Phenomena 404:132306.
Shlien, A. and D. Malkin (2009). “Copy number variations and cancer.” Genome Medicine 1 (6): 62.
Shridhar, K., G. K. Walia, A. Aggarwal, S. Gulati, A. Geetha, D. Prabhakaran, P. K. Dhillon and P. Rajaraman (2016). “DNA methylation markers for oral pre-cancer progression: A critical review.” Oral oncology 53:1-9.
Slomovic, S., D. Laufer, D. Geiger and G. Schuster (2005). “Polyadenylation and degradation of human mitochondrial RNA: the prokaryotic past leaves its mark.” Molecular and cellular biology 25 (15): 6427-6435.
Snoek, J., H. Larochelle and R. P. Adams (2012). “Practical bayesian optimization of machine learning algorithms.” Advances in neural information processing systems 25.
Stratton, M. R., P. J. Campbell and P. A. Futreal (2009). “The cancer genome.” Nature 458 (7239): 719-724.
Vacca, D., A. Fiannaca, F. Tramuto, V. Cancila, L. La Paglia, W. Mazzucco, A. Gulino, M. La Rosa, C. M. Maida and G. Morello (2022). “Direct RNA nanopore sequencing of SARS-COV-2 extracted from critical material from swabs.” Life 12 (1): 69.
Walter, R., P. Rozynek, S. Casjens, R. Werner, F. Mairinger, E. Speel, A. Zur Hausen, S. Meier, J. Wohlschlaeger and D. Theegarten (2018). “Methylation of LIRE1, RARB, and RASSF1 function as possible biomarkers for the differential diagnosis of lung cancer.” PloS one 13 (5): e0195716.
Workman, R. E., A. D. Tang, P. S. Tang, M. Jain, J. R. Tyson, R. Razaghi, P. C. Zuzarte, T. Gilpatrick, A. Payne and J. Quick (2019). “Nanopore native RNA sequencing of a human poly (A) transcriptome.” Nature methods 16 (12): 1297-1305.
Yamashita, K., K. Hosoda, N. Nishizawa, H. Katoh and M. Watanabe (2018). “Epigenetic biomarkers of promoter DNA methylation in the new era of cancer treatment.” Cancer science 109 (12): 3695-3706.
Yu, Y., X. Si, C. Hu and J. Zhang (2019). “A review of recurrent neural networks: LSTM cells and network architectures.” Neural computation 31 (7): 1235-1270.
Zhao, L., H. Zhang, M. V. Kohnen, K. V. Prasad, L. Gu and A. S. Reddy (2019). “Analysis of transcriptome and epitranscriptome in plants using PacBio Iso-Seq and nanopore-based direct RNA sequencing.” Frontiers in genetics 10:253.

We claim:

1. A system for providing a selective sequencing service over a communications network, the system comprising a frontend and a backend;
the frontend configured and adapted to:
a) collect a DNA/RNA read comprising a multiplicity of signals from one or more sequencers, the multiplicity of signals comprising a first electrical signal, a second electrical signal, and a third electrical signal,
b) trim each of the first electrical signal, the second electrical signal, and the third electrical signal, respectively, to form a trimmed signal set comprising a first trimmed signal, a second trimmed signal, and a third trimmed signal,
c) multiplex the trimmed signal set to form a multiplexed signal batch packet,
d) compress the multiplexed signal batch packet to form a compressed signal batch packet, and
e) transmit the compressed signal batch packet over the communications network to the backend; and
f) command the one or more sequencers to eject unwanted DNA/RNA according to a prediction result returned from the backend;
the backend configured and adapted to:
g) receive the compressed signal batch packet over the communications network from the frontend,
h) decompress the compressed signal batch packet to recover the multiplexed signal batch packet,
i) demultiplex the multiplexed signal batch packet to recover the trimmed signal set,
j) extract the trimmed signal set to recover the first trimmed signal, the second trimmed signal, and the third trimmed signal,
k) process each of the first trimmed signal, the second trimmed signal, and the third trimmed signal, respectively, to create an intermediate result set comprising a first intermediate result corresponding to the first electrical signal, a second intermediate result corresponding to the second electrical signal, and a third intermediate result corresponding to the third electrical signal,
l) analyze the intermediate result set to determine the prediction result of keep or eject for the DNA/RNA read, and
m) return the prediction result to the frontend over the communications network.

2. The system according to claim 1, the one or more sequencers comprising one or more sequencers, each producing one or more respective nanopore sequencer electrical signals.

3. The system according to claim 2, the backend comprising one or more artificial intelligent models trained for determining an origin of the DNA/RNA read from a sequencer electrical signal.

4. The system according to claim 3, the communications network comprising at least one of a local network running on a single computer, a local network running between two or more computers, a private network running between two or more computers, or a virtual private network segment network running between two or more computers.

5. The system according to claim 3, the communications network comprising a public internet segment.

6. The system according to claim 4, the communications network comprising a public internet segment.

7. The system according to claim 6, the one or more sequencers comprising a single sequencer configured and adapted to produce each of the first electrical signal, the second electrical signal, and the third electrical signal, respectively, in parallel.

8. The system according to claim 6, the one or more sequencers comprising a multiplicity of sequencers, each connected to a respective flowcell.

9. The system according to claim 8, the multiplicity of sequencers, comprising a first sequencer, a second sequencer, and a third sequencer, respectively configured and adapted to produce each of the first electrical signal, the second electrical signal, and the third electrical signal, respectively, in parallel, and the frontend configured and adapted to multiplex a multiplicity of concurrent reads together in the multiplexed signal batch packet.

10. The system according to claim 6, the DNA/RNA read comprising time series electrical current signals.

11. The system according to claim 9, wherein a number of concurrent reads per flow cell is less than or equal to 10.

12. A neural network for determining an origin of a DNA/RNA read from a nanopore sequencer electrical signal, the neural network comprising:
an input layer;
a first LSTM layer;
a first dropout layer;
a second LSTM layer;
a second dropout layer;
a fully-connected layer;
a ReLu layer;
a Softmax layer; and
a classification layer.

13. The neural network according to claim 12, wherein the neural network is trained based on collected nanopore sequencing signals.

14. The neural network according to claim 13, wherein the first LSTM layer comprises 128 neurons and the second LSTM layer comprises 64 neurons.

15. The neural network according to claim 14, wherein the first dropout layer, the second dropout layer, or both have a dropout probability set to about 0.25 to inhibit the neural network from overfitting.

16. The neural network according to claim 15, wherein the neural network is configured and adapted to expand itself after processing a specified number of signals.

17. The neural network according to claim 16, wherein the neural network is optimized with hyperparameter tuning based on a Bayesian Optimization method.

18. The neural network according to claim 17, wherein the hyperparameter tuning is automated to increase accuracy of the neural network.

19. The neural network according to claim 13, wherein a majority of the collected sequencing electrical signals used for training have a sample signal length between about 300 nucleotides and about 500 nucleotides.

20. A system for providing a selective sequencing service over a communications network, the system comprising a frontend and a backend;
the frontend configured and adapted to:
a) collect a DNA/RNA read comprising a multiplicity of signals from one or more sequencers, the multiplicity of signals comprising a first electrical signal, a second electrical signal, and a third electrical signal,
b) trim each of the first electrical signal, the second electrical signal, and the third electrical signal, respectively, to form a trimmed signal set comprising a first trimmed signal, a second trimmed signal, and a third trimmed signal,
c) multiplex the trimmed signal set to form a multiplexed signal batch packet,
d) compress the multiplexed signal batch packet to form a compressed signal batch packet, and
e) transmit the compressed signal batch packet over the communications network to the backend, and
f) command the one or more sequencers to eject unwanted DNA/RNA according to a prediction result returned from the backend;
the backend configured and adapted to:
g) receive the compressed signal batch packet over the communications network from the frontend,
h) decompress the compressed signal batch packet to recover the multiplexed signal batch packet,
i) demultiplex the multiplexed signal batch packet to recover the trimmed signal set,
j) extract the trimmed signal set to recover the first trimmed signal, the second trimmed signal, and the third trimmed signal,
k) process each of the first trimmed signal, the second trimmed signal, and the third trimmed signal, respectively, to create an intermediate result set comprising a first intermediate result corresponding to the first electrical signal, a second intermediate result corresponding to the second electrical signal, and a third intermediate result corresponding to the third electrical signal,
l) analyze the intermediate result set to determine the prediction result of accept or reject for the DNA/RNA read, and
m) return the prediction result to the frontend over the communications network;
the one or more sequencers comprising one or more sequencers, each producing one or more respective sequencer electrical signals;
the backend comprising one or more neural networks trained for determining an origin of the DNA/RNA read from a nanopore sequencer electrical signal;
the communications network comprising a private network or virtual private network segment, and a public internet segment;
the one or more sequencers comprising a first nanopore sequencer, a second sequencer, and a third sequencer, respectively configured and adapted to produce each of the first electrical signal, the second electrical signal, and the third electrical signal, respectively, in parallel, and the frontend configured and adapted to multiplex a number of concurrent reads together in the multiplexed signal batch packet;
the number of pores for one flow cell being less than or equal to 126 for a Flongle flowcell, less than or equal to 512 for a Minion flowcell, or less than or equal to 2675 for a PromethION or any other flowcell;
the DNA/RNA read comprising time series electrical current data;
the neural network comprising:
an input layer,
a first LSTM layer,
a first dropout layer,
a second LSTM layer,
a second dropout layer,
a fully-connected layer,
a ReLu layer,
a softmax layer, and
a classification layer;
wherein the neural network is trained based on collected nanopore sequencing signals;
wherein the first LSTM layer comprises 128 neurons and the second LSTM layer comprises 64 neurons;
wherein the first dropout layer, the second dropout layer, or both have a dropout probability set to about 0.25 to inhibit the neural network from overfitting;
wherein the neural network is configured and adapted to expand after processing a specified number of signals;
wherein the neural network is optimized with hyperparameter tuning based on a Bayesian optimization method;
wherein the hyperparameter tuning is automated; and
wherein a majority of the collected nanopore sequencing signals used for training have a sample signal length between about 400 nucleotides and about 800 nucleotides.
